(19) United States
(12) Patent Application Publication
(10) Pub. No.: US 2020/0171086 A1
(43) Pub. Date: Jun. 4, 2020

(54) Methods for Treating Lipid-Related Diseases Including Xanthomas, Carotid Artery Stenoses, and Cerebral Atherosclerosis

(71) Applicant: HDL Therapeutics, Inc., Vero Beach, FL (US)

(72) Inventors: Hollis Bryan Brewer, JR., Potomac, MD (US); Michael M. Matin, Short Hills, NJ (US)

(21) Appl. No.: 16/698,193

(22) Filed: Nov. 27, 2019

Related U.S. Application Data
(63) Continuation-in-part of application No. 16/409,543, filed on May 10, 2019.
Application No. 16/409,543 is a continuation-in-part of application No. 15/909,765, filed on Mar. 1, 2018.
Application No. 15/909,765 is a continuation-in-part of application No. 15/876,808, filed on Jan. 22, 2018.
Application No. 15/876,808 is a continuation-in-part of application No. 16/225,210, filed on Dec. 19, 2018.
Application No. 16/225,210 is a continuation-in-part of application No. 16/198,672, filed on Nov. 21, 2018.
Application No. 16/198,672 is a continuation-in-part of application No. 16/046,830, filed on Jul. 26, 2018.
Application No. 16/046,830 is a continuation-in-part of application No. 15/909,765, filed on Mar. 1, 2018.
(60) Provisional application No. 62/773,388, filed on Nov. 30, 2018.
(60) Provisional application No. 62/700,804, filed on Jul. 19, 2018.
(60) Provisional application No. 62/670,615, filed on May 11, 2018.
(60) Provisional application No. 62/537,581, filed on Jul. 27, 2017.
(60) Provisional application No. 62/516,100, filed on Jun. 6, 2017.
(60) Provisional application No. 62/465,262, filed on Mar. 1, 2017.
(60) Provisional application No. 62/516,100, filed on Jun. 6, 2017.
(60) Provisional application No. 62/465,262, filed on Mar. 1, 2017.
(60) Provisional application No. 62/449,416, filed on Jan. 23, 2017.
(60) Provisional application No. 62/611,098, filed on Dec. 28, 2017.
(60) Provisional application No. 62/589,919, filed on Nov. 22, 2017.
(60) Provisional application No. 62/537,581, filed on Jul. 27, 2017.

(51) Int. Cl. A61K 35/16; A61P 9/10

(52) U.S. Cl. CPC A61K 35/16; A61P 9/10

(57) ABSTRACT

Systems and methods for treating lipid-related diseases including xanthomas, carotid artery stenosis (CAS), and cerebral atherosclerosis (CA) and their complications use direct visualization or imaging techniques to assess the state of the anatomy at issue. A high density lipoprotein composition is made and administered to a patient in order to treat those lipid-related diseases. The administration is continued for a predetermined time or until certain anatomical changes are observed based on imaging, biomarker, or biopsy analysis.

### CROSS-REFERENCE

The present application relies on U.S. Patent Provisional Application No. 62/773,388, entitled “Methods for Treating Cholesterol Related Diseases” and filed on Nov. 30, 2018, for priority.

The present application is also a continuation-in-part application of U.S. patent application Ser. No. 16/409,543, entitled “Methods for Prophylactically Preventing, Slowing the Progression of, or Treating Cerebral Amyloid Angiopathy, Alzheimer's Disease and/or Acute Stroke” and filed on May 10, 2019, which relies on U.S. Provisional Patent Application No. 62/700,804, entitled “Methods for Prophylactically Preventing, Slowing the Progression of, or Treating Cerebral Amyloid Angiopathy, Alzheimer's Disease and/or Acute Stroke” and filed on Jul. 19, 2018 and U.S. Provisional Patent Application No. 62/670,615, of the same title and filed on May 11, 2018, for priority.

U.S. patent application Ser. No. 16/409,543 is also a continuation-in-part application of U.S. patent application Ser. No. 15/909,765, entitled “Methods for Prophylactically Preventing, Slowing the Progression of, or Treating Alzheimer's Disease” and filed on Mar. 1, 2018, which relies on U.S. Provisional Patent Application No. 62/537,581, entitled “Methods for Treating Cholesterol-Related Diseases” and filed on Jul. 27, 2017, U.S. Provisional Patent Application No. 62/516,100, entitled “Methods for Treating Cholesterol-Related Diseases” and filed on Jun. 6, 2017, and U.S. Provisional Patent Application No. 62/465,262, entitled “Method for Treating Familial Hypercholesterolemia” and filed on Mar. 1, 2017, for priority.

U.S. patent application Ser. No. 15/909,765 is also a continuation-in-part application of U.S. patent application Ser. No. 15/876,808, entitled “Methods for Treating Cholesterol-Related Diseases”, and filed on Jan. 22, 2018, which, in turn, relies on U.S. Provisional Patent Application No. 62/516,100, entitled “Methods for Treating Cholesterol-Related Diseases” and filed on Jun. 6, 2017, U.S. Provisional Patent Application No. 62/465,262, entitled “Method for Treating Familial Hypercholesterolemia” and filed on Mar. 1, 2017, and U.S. Provisional Patent Application No. 62/449,416, entitled “Method for Treating Familial Hypercholesterolemia” and filed on Jan. 23, 2017, for priority.

The present application is also a continuation-in-part application of U.S. patent application Ser. No. 16/225,210, entitled “Methods for Preserving and Administering Pre-Beta High Density Lipoprotein Extracted from Human Plasma” and filed on Dec. 19, 2018, which relies on U.S. Provisional Patent Application No. 62/611,098, entitled “Methods for Treating Cholesterol-Related Diseases” and filed on Dec. 28, 2017, for priority.

The present application is also a continuation-in-part application of U.S. patent application Ser. No. 16/198,672, entitled “Systems and Methods for Priming Fluid Circuits of a Plasma Processing System” and filed on Nov. 21, 2018, which relies on U.S. Provisional Patent Application No. 62/589,919, entitled “Systems and Methods for Causing Regression of Arterial Plaque” and filed on Nov. 22, 2017, for priority.

The present application is also a continuation-in-part application of U.S. patent application Ser. No. 16/046,830, entitled “Methods for Treating Cholesterol-Related Diseases Using Administered Solutions Having Increased Pre-Beta HDL Particles” and filed on Jul. 26, 2018, which relies on U.S. Provisional Patent Application No. 62/537,581, entitled “Method for Treating Cholesterol-Related Diseases” and filed on Jul. 27, 2017, for priority.

U.S. patent application Ser. No. 16/046,830 is also a continuation-in-part application of U.S. patent application Ser. No. 15/909,765, entitled “Methods for Prophylactically Preventing, Slowing the Progression of, or Treating Alzheimer's Disease”, and filed on Mar. 1, 2018.

The above-mentioned applications are all incorporated herein by reference in their entirety.

### FIELD

The present specification generally relates to systems, apparatuses and methods for treating xanthomas, carotid artery stenoses, and cerebral atherosclerosis by the extracorporeal treatment of blood plasma using either a single solvent or multiple solvents. More particularly, the systems and methods of the present specification provide for a successively repeatable treatment for selective removal of lipids from HDL particles to create a modified HDL particle while leaving LDL particles substantially intact in order to treat xanthomas, carotid artery stenoses, and cerebral atherosclerosis.

### BACKGROUND

Xanthomas are characterized by a build-up of fats under the skin surface. The condition is recognized by the deposition of yellowish cholesterol-rich material in which lipids accumulate in foam cells of different sizes within the skin. Xanthomas are commonly located in areas such as on or around the eyelids, over the joints, in the tendons of the hands, feet, and heels, and in the buttocks, among other body parts. The condition is common among older adults and people with high concentrations of blood lipids. Xanthomas are also a sign of medical conditions such as high blood cholesterol levels and inherited metabolic disorders, such as familial hypercholesterolemia, though they can also occur in patients with normal lipid levels. Xanthomas are usually diagnosed by visual inspections of the xanthoma. Xanthomas are commonly removed via surgery. However, surgery does not address an underlying medical condition that may be resulting in a repeated xanthoma. Moreover, surgery is costly, can be painful, and often leaves scars on the skin surface. In some instances, some xanthomas cannot be readily addressed via surgery, such as those near or around the eyelids. In cases where the xanthomas are a result of medical conditions such as familial hypercholesterolemia or severe secondary hyperlipidemia, it may be beneficial to address the lipids that deposit to form the xanthomas. However, existing treatments for lipids have not been shown to be effective for treating xanthomas.

Acute strokes, also common in the elderly, may also be caused by an increase in cholesterol levels and metabolic disorders such as familial hypercholesterolemia. Cholesterol is synthesized by the liver or obtained from dietary sources. Low-density lipoprotein (LDL) is responsible for transferring cholesterol from the liver to tissues at different sites in the body. However, if LDL collects on the arterial walls, it undergoes oxidation caused by oxygen free radicals liberated from the body's chemical processes and interacts deleteriously with the blood vessels. The modified LDL causes white blood cells in the immune system to gather at the arterial walls, forming a fatty substance called plaque and injuring cellular layers that line blood vessels. The modified oxidized LDL also reduces the level of nitric oxide, which is responsible for relaxing the blood vessels and thereby allowing the blood to flow freely. As this process continues, the arterial walls slowly constrict, resulting in hardening of the arteries and thereby reducing blood flow. The gradual build-up of plaque can result in blockage of a carotid vessel and ultimately in a stroke. The underlying condition for this type of obstruction is the development of fatty deposits lining the vessel walls. It is known that at least 2.7% of men and women over the age of 18 in the United States have a history of stroke. Prevalence of stroke is also known to be higher with increasing age. With the increase in the aging population, the prevalence of stroke survivors is projected to increase, especially among elderly women. A considerable portion of all strokes (at least 87%) are ischemic in nature.

Ischemic stroke occurs when there is a blockage in an artery leading to the brain, and may be a secondary condition caused by a hemorrhagic stroke. Among the different types of ischemic stroke, the most common are thrombotic and embolic. A thrombotic stroke occurs when diseased or damaged cerebral arteries become blocked by the formation of a blood clot within the brain. Sometimes the blockage is in one of the brain's larger blood-supplying arteries such as the carotid artery or middle cerebral artery. The risk of carotid artery stenosis increases significantly with abnormal (high) lipid levels or high cholesterol as plaque usually builds up in the carotid arteries until the occurrence of a stroke. During a medical examination, a physician may be able to diagnose a possibility of carotid artery disease based on abnormal sounds, called bruits, with the help of a stethoscope. Physicians may also use tests to diagnose carotid artery stenosis, such as carotid ultrasound, magnetic resonance angiography (MRA), computerized tomography angiography (CTA), and cerebral angiography (carotid angiogram). Surgical procedures may be performed to open the carotid artery and remove plaques. Known procedures include carotid endarterectomy (CEA) and carotid artery stenting (CAS). In the latter, a stent is placed in the artery and expanded to hold the artery open. However, these are invasive procedures and involve risks. Cerebral arteries are not stented and surgical procedures on cerebral arteries are also invasive and involve risks.

Further, it has been shown that hypercholesterolemia and inflammation are two dominant mechanisms implicated in the development of atherosclerosis. There is significant overlap between vascular risk factors for both Alzheimer's disease and atherosclerosis. Inflammation has been implicated in Alzheimer's disease pathogenesis and it is suggested that abnormalities in cholesterol homeostasis may have a role as well. In addition, many of the contributory factors in atherogenesis also contribute to Alzheimer's disease. Specifically, in cell cultures, increased and decreased cholesterol levels promote and inhibit the formation of beta amyloid (Aβ) from amyloid precursor protein (APP), respectively. Thus, the use of treatments with proven effects on the process of atherosclerosis may be one method for treating the progression of Alzheimer's disease.

In contrast to LDL, high plasma high-density lipoprotein (HDL) levels are desirable because they play a major role in “reverse cholesterol transport”, where the excess cholesterol is transferred from tissue sites to the liver where it is eliminated. Optimal total cholesterol levels are 200 mg/dl or below with a LDL cholesterol level of 160 mg/dl or below and a HDL cholesterol level of 45 mg/dl for men and 50 mg/dl for women. Lower LDL levels are recommended for individuals with a history of elevated cholesterol, atherosclerosis, or coronary artery disease. High levels of LDL increase the lipid content in coronary arteries resulting in formation of lipid filled plaques that are vulnerable to rupture. On the other hand, HDL has been shown to decrease the lipid content in the lipid filled plaques, reducing the probability of rupture. In the last several years, clinical trials of LDL lowering drugs have definitively established that reductions in LDL are associated with a 30-45% decrease in clinical cardiovascular disease (CVD) events. CVD events include events occurring in diseases such as homozygous familial hypercholesterolemia (HoFH), heterozygous familial hypercholesterolemia (HeFH), and peripheral arterial disease. Despite lowered LDL, however, many patients continue to have cardiac events. Low levels of HDL are often present in high risk subjects with CVD, and epidemiological studies have identified HDL as an independent risk factor that modulates CVD risk. In addition to epidemiologic studies, other evidence suggests that raising HDL would reduce the risk of CVD. There has been increasing interest in changing plasma HDL levels by dietary, pharmacological or genetic manipulations as a potential strategy for the treatment of CVD including HoFH, HeFH, ischemic stroke, coronary artery disease (CAD), acute coronary syndrome (ACS), and peripheral arterial disease and for treating the progression of Alzheimer's disease.

The protein component of LDL, known as apolipoprotein-B (ApoB), and its products, comprise atherogenic elements. Elevated plasma LDL levels and reduced HDL levels are recognized as primary causes of coronary and carotid artery disease. ApoB is in highest concentrations in LDL particles and is not present in HDL particles. Apolipoprotein A-I (ApoA-I) and apolipoprotein A-II (ApoA-II) are found in HDL particles. Other apolipoproteins, such as apolipoprotein-C(ApoC) and its subtypes (C-I, C-II and C-III), apolipoprotein-D (ApoD), and apolipoprotein-E (ApoE) are also found in HDL particles. ApoC and ApoE are also observed in LDL particles.

Numerous major classes of HDL particles including HDL2b, HDL2a, HDL3a, HDL3b and HDL3 have been reported. Various forms of HDL particles have been described on the basis of electrophoretic mobility on agarose as two major populations: a major fraction with α-HDL mobility; and, a minor fraction with migration similar to very-low-density lipoprotein (VLDL). This latter fraction has been called pre-β HDL and these particles represent the most efficient HDL particle subclass for inducing cellular cholesterol efflux.

The HDL lipoprotein particles are comprised of apolipoprotein A-I (ApoA-I), phospholipids and cholesterol. The pre-β HDL particles are considered to be the first acceptors of cellular free cholesterol and are essential in eventually transferring free and esterified cholesterol to α-HDL. Pre-β HDL particles may transfer cholesterol to α-HDL or be converted to α-HDL. The alpha HDL transfers cholesterol to the liver, where excess cholesterol can be removed from the body.

HDL levels are inversely correlated with atherosclerosis and carotid artery disease. Once cholesterol-carrying α-HDL reaches the liver, the α-HDL particles divest of the cholesterol and transfer the free cholesterol to the liver. The α-HDL particles (divested of cholesterol) are subsequently converted to pre-β HDL particles and exit the liver, which then serve to pick up additional cholesterol within the body and are converted back to α-HDL, thus repeating the cycle. Accordingly, what is needed is a method to decrease or remove cholesterol from these various HDL particles, especially the α-HDL particles, so that they are available to remove additional cholesterol from cells.

Hyperlipidemia (or abnormally high concentration of lipids in the blood) may be treated by changing a patient's diet. However, diet as a primary mode of therapy requires a major effort on the part of patients, physicians, nutritionists, dietitians, and other health care professionals and thus undesirably taxes the resources of health professionals. Another negative aspect of this therapy is that its success does not rest exclusively on diet. Rather, success of dietary therapy depends upon a combination of social, psychological, economic, and behavioral factors. Thus, therapy based only on correcting flaws within a patient's diet is not always successful.

In instances when dietary modification has been unsuccessful, drug therapy has been used as adjunctive therapy. Such therapy has included the use of commercially available hypolipidemic drugs administered alone or in combination with other therapies as a supplement to dietary control. These drugs, called statins, include lovastatin, pravastatin, simvastatin, fluvastatin, atorvastatin, and cerivastatin. Statins are particularly effective for lowering LDL levels and are also effective in the reduction of triglycerides, apparently in direct proportion to their LDL-lowering effects. Statins raise HDL levels, but to a lesser extent than other anti-cholesterol drugs. Statins also increase nitric oxide, which, as described above, is reduced in the presence of oxidized LDL.

Bile acid resins, another drug therapy, work by binding with bile acid, a substance made by the liver using cholesterol as one of the primary manufacturing components. Because the drugs bind with bile acids in the digestive tract, they are then excreted with the feces rather than being absorbed into the body. The liver, as a result, must take more cholesterol from the circulation to continue constructing bile acids, resulting in an overall decrease in LDL levels. Nicotinic acid, or niacin, also known as vitamin B3, is effective in reducing triglyceride levels and raising HDL levels higher than any other anti-cholesterol drug. Nicotinic acid also lowers LDL-cholesterol.

Fibric acid derivatives, or fibrates, are used to lower triglyceride levels and increase HDL when other drugs ordinarily used for these purposes, such as niacin, are not effective. Probucol lowers LDL-cholesterol levels, however, it also lowers HDL levels. It is generally used for certain genetic disorders that cause high cholesterol levels, or in cases where other cholesterol-lowering drugs are ineffective or cannot be used.

Proprotein convertase subtilisin/kexin type 9 (PCSK9) inhibitors lower LDL-cholesterol levels via increasing the cellular level of LDL receptors that reside in the liver.

Hypolipidemic drugs have had varying degrees of success in reducing blood lipids, however, none of the hypolipidemic drugs successfully treats all types of hyperlipidemia. While some hypolipidemic drugs have been fairly successful, the medical community has found little conclusive evidence that hypolipidemic drugs cause regression of atherosclerosis. In addition, all hypolipidemic drugs have undesirable side effects. As a result of the lack of success of dietary control, drug therapy and other therapies, atherosclerosis remains a major cause of death in many parts of the world.

New therapies have been used to reduce the amount of lipid in patients for whom drug and diet therapies were not sufficiently effective. For example, extracorporeal procedures like plasmapheresis and LDL-apheresis have been employed and are shown to be effective in lowering LDL.

Plasmapheresis therapy, or plasma exchange therapy, involves replacing a patient's plasma with donor plasma or more usually a plasma protein fraction. Plasmapheresis is a process whereby the blood plasma is removed from blood cells by a cell separator. The separator works either by spinning the blood at high speed to separate the cells from the fluid or by passing the blood through a membrane with pores so small that only the fluid component of the blood can pass through. The cells are returned to the person undergoing treatment, while the plasma is discarded and replaced with other fluids.

This treatment has resulted in complications due to the introduction of foreign proteins and transmission of infectious diseases. Further, plasmapheresis has the disadvantage of non-selective removal of all serum lipoproteins, such as VLDL, LDL, and HDL. Moreover, plasmapheresis can result in several side effects including allergic reactions in the form of fever, chills, rash, and possibly anaphylaxis.

As described above, it is not desirable to remove HDL, which is secreted from both the liver and the intestine as nascent, disk-shaped particles that contain cholesterol and phospholipids. HDL is believed to play a role in reverse cholesterol transport, which is the process by which excess cholesterol is removed from tissues and transported to the liver for reuse or disposal in the bile.

In contrast to plasmapheresis, the LDL-apheresis procedure selectively removes ApoB containing cholesterol, such as LDL, while retaining HDL.

Several methods for LDL-apheresis have been developed. These techniques include absorption of LDL in heparin-agarose beads, the use of immobilized LDL-antibodies, cascade filtration absorption to immobilize dextran sulfate, and LDL precipitation at low pH in the presence of heparin. Each method described above is effective in removing LDL. This treatment process has disadvantages, however, including the failure to positively affect HDL or to cause a metabolic shift that can enhance atherosclerosis and other cardiovascular diseases. LDL-apheresis, as its name suggests, merely treats LDL in patients with severe hyperlipidemia.

Yet another method of achieving a reduction in plasma cholesterol in homozygous familial hypercholesterolemia, heterozygous familial hypercholesterolemia, and patients with acquired hyperlipidemia is an extracorporeal lipid elimination process, referred to as cholesterol apheresis. In cholesterol apheresis, blood is withdrawn from a patient, the plasma is separated from the blood, and the plasma is mixed with a solvent mixture. The solvent mixture extracts lipids from the plasma. Thereafter, the delipidated plasma is recombined with the patient's blood cells and returned to the patient. Using this procedure, however, results in a modification of the LDL particles, such that the modified LDL particles could result in increased intensity of xanthomas or carotid stenosis. At the same time, this process also may result in further delipidation of the HDL particles.

Conventional extracorporeal delipidation processes, however, are directed toward the concurrent delipidation of LDL and HDL. This process can have a number of disadvantages, mainly in that delipidated LDL tends to aggregate and subsequently cause an increase in heart disease conditions, rather than decrease. In addition, extracorporeal systems are designed to subject body fluid volumes to substantial processing, possibly through multiple stage solvent exposure and extraction steps.

Vigorous multi-stage solvent exposure and extraction can have several drawbacks. It may be difficult to remove a sufficient amount of solvents from the delipidated plasma in order for the delipidated plasma to be safely returned to a patient.

Hence, existing apheresis and extracorporeal systems for treatment of plasma constituents suffer from a number of disadvantages that limit their ability to be used in clinical applications. A need exists for improved systems, apparatuses and methods capable of removing lipids from blood components in order to provide treatments and preventative measures for chronic diseases. Methods have also been provided to selectively remove lipid from HDL particles and thereby create modified HDL particles with increased capacity to accept cholesterol.

While the methods to selectively delipidate HDL particles overcome several of the limitations stated above, what is also needed is a method to selectively remove lipid from HDL particles and thereby create modified HDL particles with increased capacity to accept cholesterol, without substantially affecting LDL particles, in chronic diseases. What is also needed is a method to successively monitor the effectiveness of the modified HDL particles in accepting cholesterol in order to monitor the progress of a treatment using imaging techniques such as CT angiography. Additionally, what is needed is a method to treat xanthomas and carotid stenosis and prevent the onset of stroke.

### SUMMARY

The following embodiments and aspects thereof are described and illustrated in conjunction with systems, tools and methods, which are meant to be exemplary and illustrative, not limiting in scope.

The present specification discloses a method for treatment of at least one xanthoma in a patient wherein dyslipidemia is diagnosed as an underlying cause for the at least one xanthoma, comprising: monitoring changes in at least one of size, volume, location, or composition of the at least one xanthoma; determining if lipid-containing degenerative material is present in one or more blood vessels; based on the determination of lipid-containing degenerative material and the monitoring of changes in at least one of size, volume, location, or composition of the at least one xanthoma, determining a treatment protocol for said at least one xanthoma, wherein the treatment protocol comprises administering to the patient a high density lipoprotein composition derived from mixing a blood fraction with a lipid removing agent; and administering to the high density lipoprotein composition in accordance with the treatment protocol.

Optionally, the method further comprises monitoring changes in the one or more blood vessels in the patient in order to determine if lipid-containing degenerative material is present in the one or more blood vessels.

Optionally, the high density lipoprotein composition is derived by: obtaining the blood fraction from the patient; mixing said blood fraction with the lipid removing agent to yield modified high-density lipoproteins; separating said modified high-density lipoproteins; and delivering said modified high-density lipoproteins to said patient. The modified high density lipoproteins may have an increased concentration of pre-beta high density lipoprotein relative to high density lipoproteins from the blood fraction prior to mixing.

Optionally, the method further comprises: connecting the patient to a device for withdrawing blood; withdrawing blood from the patient; and separating blood cells from the blood to yield the blood fraction containing high density lipoproteins and low density lipoproteins.

Optionally, the method further comprises repeating the administration to the patient of the high density lipoprotein composition.

Optionally, the method further comprises, after said administration to the patient of the high density lipoprotein composition, monitoring changes in the at least one xanthoma by determining a change in a hardness of the at least one xanthoma. The change may be determined by comparing a first hardness of the at least one xanthoma measured prior to said administration to a second hardness of the at least one xanthoma measured after said administration. Optionally, the method further comprises determining the first hardness and second hardness by applying a durometer to the at least one xanthoma. Optionally, the method further comprises repeating the administration to the patient of the high density lipoprotein composition until a durometer value of the second hardness is at least 10% less than a durometer value of the first hardness. Optionally, the method further comprises stopping the administration to the patient of the high density lipoprotein composition after a durometer value of the second hardness is at least 10% less than a durometer value of the first hardness.

Optionally, the method further comprises determining the size of the at least one xanthoma using at least one of a laser lines guide, a 3D scanner, or a wound measurement device.

Optionally, the method further comprises administering to the patient the high density lipoprotein composition only if a high sensitivity C reactive protein value of the patient exceeds a threshold value. The threshold value may be greater than 1 mg/dl. Optionally, the method further comprises not administering to the patient the high density lipoprotein composition if the high sensitivity C reactive protein value of the patient does not exceed the threshold value.

Optionally, the method further comprises administering to the patient a high density lipoprotein composition only if an interleukin 18 level of the patient exceeds a threshold value. The threshold value may be greater than 32 pg/ml. Optionally, the method further comprises not administering to the patient the high density lipoprotein composition if the interleukin 18 level of the patient does not exceed the threshold value.

Optionally, the method further comprises administering to the patient the high density lipoprotein composition only if a tumor necrosis factor alpha level of the patient exceeds a threshold value. The threshold value may be greater than 11. Optionally, the method further comprises not administering to the patient the high density lipoprotein composition if the tumor necrosis factor alpha level of the patient does not exceed the threshold value.

The present specification also discloses a method for treatment of carotid artery stenosis in a patient, comprising: monitoring changes in one or more blood vessels of the patient, wherein the one or more blood vessels comprise a carotid artery of the patient; determining if lipid-containing degenerative material is present in said one or more blood vessels; based on said monitoring and the determination of lipid-containing degenerative material, determining a treatment protocol for the carotid artery stenosis, wherein the treatment protocol comprises a placement of a stent in the patient and an administration to the patient of a composition derived from mixing a blood fraction of the patient with a lipid removing agent only if a blockage of the carotid artery of the patient exceeds 20% and does not exceed 70% of the carotid artery of the patient; placing said stent; and administering the high density lipoprotein composition to the patient in accordance with the treatment protocol.

Optionally, the composition is derived by: obtaining the blood fraction from the patient; mixing said blood fraction with the lipid removing agent to yield modified high-density lipoproteins; separating said modified high-density lipoproteins; and delivering said modified high-density lipoproteins to said patient. The modified high density lipoproteins may have an increased concentration of pre-beta high density lipoprotein relative to high density lipoproteins from the blood fraction prior to mixing.

Optionally, the method further comprises: connecting the patient to a device for withdrawing blood; withdrawing blood from the patient; and separating blood cells from the blood to yield the blood fraction containing high density lipoproteins and low density lipoproteins.

Optionally, the method further comprises repeating the method of treatment based on the monitoring of changes in the one or more blood vessels of the patient.

Optionally, the method further comprises repeating the administration to the patient of the high density lipoprotein composition.

Optionally, the method further comprises, after said administration to the patient of the high density lipoprotein composition, monitoring changes in the carotid artery by determining a change in an inflammation level of a portion of the carotid artery. The change in the inflammation level may be determined by using at least one of an imaging of the carotid artery, a biopsy of the carotid artery, or a measurement of a plasma biomarker level.

Optionally, the method further comprises determining a first inflammation level measured prior to the administration to the patient of the high density lipoprotein composition and determining a second inflammation level measured after the administration to the patient of the high density lipoprotein composition. Optionally, the method further comprises repeating the administration to the patient of the high density lipoprotein composition until the measured second inflammation level exceeds a threshold reduction in value relative to the measured first inflammation level. The threshold reduction in value may be 10%. Optionally, the method further comprises stopping the administration to the patient of the high density lipoprotein composition once the measured second inflammation level exceeds a threshold reduction in value relative to the measured first inflammation level. The threshold reduction in value may be 10%.

Optionally, the method further comprises administering to the patient the high density lipoprotein composition at least one time per week for a duration of 1 to 14 weeks.

Optionally, the method further comprises determining a first thickness in a wall of the carotid artery prior to the administration to the patient of the high density lipoprotein composition and determining a second thickness in the wall of the carotid artery measured after the administration to the patient of the high density lipoprotein composition. Optionally, the method further comprises repeating the administration to the patient of the high density lipoprotein composition until the determined second thickness exceeds a threshold reduction in value relative to the determined first thickness. The threshold reduction in value may be 15%. Optionally, the method further comprises stopping the administration to the patient of the high density lipoprotein composition once the determined second thickness exceeds a threshold reduction in value relative to the determined first thickness. The threshold reduction in value may be 15%.

The present specification also discloses a method for treatment of cerebral atherosclerosis in a patient, comprising: monitoring changes in one or more blood vessels of the patient, wherein the one or more blood vessels comprise one or more cerebral arteries of the patient; determining if lipid-containing degenerative material is present in said one or more blood vessels; based on said monitoring and the determination of lipid-containing degenerative material, determining a treatment protocol for the cerebral atherosclerosis, wherein the treatment protocol comprises a placement of a stent in the patient and an administration to the patient of a composition derived from mixing a blood fraction of the patient with a lipid removing agent only if a blockage of the one or more cerebral arteries of the patient exceeds 20% and does not exceed 70% of the one or more cerebral arteries of the patient; placing said stent; and administering said composition only if a blockage of the one or more cerebral arteries of the patient exceeds 20% and does not exceed 70% of the one or more cerebral arteries of the patient.

Optionally, the composition is derived by: obtaining the blood fraction from the patient; mixing said blood fraction with the lipid removing agent to yield modified high-density lipoproteins; separating said modified high-density lipoproteins; and delivering said modified high-density lipoproteins to said patient.

The modified high density lipoproteins may have an increased concentration of pre-beta high density lipoprotein relative to high density lipoproteins from the blood fraction prior to mixing.

Optionally, the method further comprises: connecting the patient to a device for withdrawing blood; withdrawing blood from the patient; and separating blood cells from the blood to yield the blood fraction containing high density lipoproteins and low density lipoproteins.

Optionally, the method further comprises repeating the method of treatment based on the monitoring of changes in the one or more blood vessels of the patient.

Optionally, the method further comprises repeating the administration to the patient of the high density lipoprotein composition.

Optionally, the method further comprises, after said administration to the patient of the high density lipoprotein composition, monitoring changes in the one or more cerebral arteries by determining a change in an inflammation level of a portion of the one or more cerebral arteries. The change in the inflammation level may be determined by using at least one of an imaging of the one or more cerebral arteries, a biopsy of the one or more cerebral arteries, or a measurement of a plasma biomarker level.

Optionally, the further comprises determining a first inflammation level measured prior to the administration to the patient of the high density lipoprotein composition and determining a second inflammation level measured after the administration to the patient of the high density lipoprotein composition.

Optionally, the method further comprises repeating the administration to the patient of the high density lipoprotein composition until the measured second inflammation level exceeds a threshold reduction in value relative to the measured first inflammation level. The threshold reduction in value may be 10%. Optionally, the method further comprises stopping the administration to the patient of the high density lipoprotein composition once the measured second inflammation level exceeds a threshold reduction in value relative to the measured first inflammation level. The threshold reduction in value may be 10%.

Optionally, the method further comprises administering to the patient the high density lipoprotein composition at least one time per week for a duration of 1 to 14 weeks.

Optionally, the method further comprises determining a first thickness in a wall of the one or more cerebral arteries prior to the administration to the patient of the high density lipoprotein composition and determining a second thickness in the wall of the one or more cerebral arteries measured after the administration to the patient of the high density lipoprotein composition. Optionally, the method further comprises repeating the administration to the patient of the high density lipoprotein composition until the determined second thickness exceeds a threshold reduction in value relative to the determined first thickness. The threshold reduction in value may be 15%. Optionally, the method further comprises stopping the administration to the patient of the high density lipoprotein composition once the determined second thickness exceeds a threshold reduction in value relative to the determined first thickness. The threshold reduction in value may be 15%.

The aforementioned and other embodiments of the present specification shall be described in greater depth in the drawings and detailed description provided below.

### BRIEF DESCRIPTION OF THE DRAWINGS

These and other features and advantages of the present specification will be appreciated, as they become better understood by reference to the following detailed description when considered in connection with the accompanying drawings, wherein:

FIG. 1A is a flow chart delineating the steps of treating diseases, such as xanthomas and carotid artery stenosis (CAS), using the treatment systems and methods in accordance with embodiments of the present specification;

FIG. 1B is a flow chart delineating the steps of treating xanthomas, in accordance with embodiments of the present specification;

FIG. 1C is a flow chart delineating the steps of treating CAS, in accordance with embodiments of the present specification;

FIG. 1D is a flow chart delineating the steps of treating cerebral atherosclerosis (CA), in accordance with embodiments of the present specification;

FIG. 2 is a schematic representation of a plurality of components used in accordance with some embodiments of the present specification to achieve the processes disclosed herein;

FIG. 3 is a pictorial illustration of an exemplary embodiment of a configuration of a plurality of components used in accordance with some embodiments of the present specification to achieve the processes disclosed herein;

FIG. 4 is an illustrative representation of plaque in a carotid artery of a patient; and

FIG. 5 is an illustrative representation of plaque in a cerebral artery of a patient.

### DETAILED DESCRIPTION

The present specification relates to methods and systems for treating cholesterol-related diseases. Embodiments of the present specification monitor changes in one or more xanthomas and/or atheroma areas and volumes in a patient regularly over a period of time. Atheroma areas and volumes are monitored using known imaging techniques for lipid-containing degenerative material in stenosis. Imaging techniques for monitoring lipid-containing degenerative material include, but are not limited to, computerized tomography (CTA), intravascular ultrasound (IVUS), magnetic resonance angiography (MRA), carotid ultrasound, and intravascular optical coherence tomography (OTC).

In accordance with embodiments of the present specification, based on the results of the monitoring, treatment is provided if accumulated lipid-containing degenerative material is identified to be present. The treatment is repeated each time the atheroma areas and volumes are monitored, at pre-defined time intervals, and accumulated lipid-containing degenerative material is identified to be present. In some embodiments, a threshold may be defined, and the treatment is provided and/or continued if the accumulated lipid-containing degenerative material is identified to be above the threshold. In various embodiments, the threshold is defined based on the level of stenosis of the blood vessel being monitored. In various embodiments, patients presenting with stenosis of a carotid or cerebral artery in a range of 20%-70% are candidates and receive delipidation therapy in accordance with the systems and methods of the present specification. In some embodiments, patients presenting with less than 20% stenosis of a carotid or cerebral artery do not receive therapy and are continually monitored for changes in the level of stenosis. In some embodiments, patients presenting with greater than 70% stenosis of a carotid or cerebral artery undergo a surgical procedure for placing a stent in the affected artery and then also receive delipidation therapy. Placement of the stent addresses a systemic disease with a local intervention. The delipidation therapy is used to remove existing plaques. In some embodiments, stent placement is based on fraction flow reserves (FFR), wherein patients having an FFR of greater than 70% receive a stent.

Embodiments of the present specification treat the condition through systems, apparatuses and methods useful for removing lipids from α-high density lipoprotein (α-HDL) particles derived primarily from plasma of the patient thereby creating modified HDL particles with reduced lipid content, particularly reduced cholesterol content. Embodiments of the present specification create modified HDL particles with reduced lipid content without substantially modifying LDL particles. Embodiments of the present specification modify original α-HDL particles to yield modified HDL particles that have an increased concentration of pre-β HDL relative to the original HDL.

Further, the newly formed derivatives of HDL particles (modified HDL) are administered to the patient to enhance cellular cholesterol efflux and treat cardiovascular diseases and/or other lipid-associated diseases, including xanthomas and carotid stenosis. The regular periodic monitoring and treatment process renders the methods and systems of the present specification more effective in treating the diseases such as, but not limited to, homozygous familial hypercholesterolemia (HoFH), heterozygous familial hypercholesterolemia (HeFH), xanthomas, ischemic stroke, cerebral atherosclerosis (CA) and carotid artery stenosis (CAS), among others.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention. In the description and claims of the application, each of the words “comprise” “include” and “have”, and forms thereof, are not necessarily limited to members in a list with which the words may be associated.

It should be noted herein that any feature or component described in association with a specific embodiment may be used and implemented with any other embodiment unless clearly indicated otherwise.

The term “fluid” may be defined as fluids from animals or humans that contain lipids or lipid containing particles, fluids from culturing tissues and cells that contain lipids and fluids mixed with lipid-containing cells. For purposes of the present specification, decreasing the amount of lipids in fluids includes decreasing lipids in plasma and particles contained in plasma, including but not limited to HDL particles. Fluids include, but are not limited to: biological fluids; such as blood, plasma, serum, lymphatic fluid, cerebrospinal fluid, peritoneal fluid, pleural fluid, pericardial fluid, and various fluids of the reproductive system including, but not limited to, semen, ejaculatory fluids, follicular fluid and amniotic fluid; cell culture reagents such as normal sera, fetal calf serum or serum derived from any animal or human; and immunological reagents, such as various preparations of antibodies and cytokines from culturing tissues and cells, fluids mixed with lipid-containing cells, and fluids containing lipid-containing organisms, such as a saline solution containing lipid-containing organisms. A preferred fluid treated with the methods of the present invention is plasma.

The term “lipid” may be defined as any one or more of a group of fats or fat-like substances occurring in humans or animals. The fats or fat-like substances are characterized by their insolubility in water and solubility in organic solvents. The term “lipid” is known to those of ordinary skill in the art and includes, but is not limited to, complex lipid, simple lipid, triglycerides, fatty acids, glycerophospholipids (phospholipids), true fats such as esters of fatty acids, glycerol, cerebrosides, waxes, and sterols such as cholesterol and ergosterol.

The term “extraction solvent” may be defined as one or more solvents used for extracting lipids from a fluid or from particles within the fluid. This solvent enters the fluid and remains in the fluid until removed by other subsystems. Suitable extraction solvents include solvents that extract or dissolve lipid, including but not limited to phenols, hydrocarbons, amines, ethers, esters, alcohols, halohydrocarbons, halocarbons, and combinations thereof. Examples of suitable extraction solvents are ethers, esters, alcohols, halohydrocarbons, or halocarbons which include, but are not limited to di-isopropyl ether (DIPE), which is also referred to as isopropyl ether, diethyl ether (DEE), which is also referred to as ethyl ether, lower order alcohols such as butanol, especially n-butanol, ethyl acetate, dichloromethane, chloroform, isoflurane, sevoflurane (1,1, 1,3, 3,3-hexafluoro-2-(fluoromethoxy) propane-d3), perfluorocyclohexanes, trifluoroethane, cyclofluorohexanol, and combinations thereof.

The term “patient” refers to animals and humans, which may be either a fluid source to be treated with the methods of the present specification or a recipient of derivatives of HDL particles and or plasma with reduced lipid content.

The term “HDL particles” encompasses several types of particles defined based on a variety of methods such as those that measure charge, density, size and immuno-affinity, including but not limited to electrophoretic mobility, ultracentrifugation, immunoreactivity and other methods known to one of ordinary skill in the art. Such HDL particles include, but are not limited to, α-HDL, pre-β HDL (including pre-β1 HDL, pre-β2 HDL and pre-β3HDL), HDL2 (including HDL2a and HDL2b), HDL3, VHDL, lipoprotein A-1 (LpA-I), lipoprotein A-2 (LpA-II), and LpA-I/LpA-II (for a review see Barrans et al., Biochemica Biophysica Acta 1300; 73-85,1996). Accordingly, practice of the methods of the present invention creates modified HDL particles. These modified derivatives of HDL particles may be modified in numerous ways including but not limited to changes in one or more of the following metabolic and/or physico-chemical properties (for a review see Barrans et al., Biochemica Biophysica Acta 1300; 73-85, 1996): molecular mass (kDa); charge; diameter; shape; density; hydration density; flotation characteristics; content of cholesterol; content of free cholesterol; content of esterified cholesterol; molar ratio of free cholesterol to phospholipids; immuno-affinity; content, activity or helicity of one or more of the following enzymes or proteins: apolipoprotein A-I (ApoA-I), apolipoprotein A-II (ApoA-II), apolipoprotein D (ApoD), apolipoprotein E (ApoE), apolipoprotein J (ApoJ), apolipoprotein A-IV (ApoA-IV), cholesterol ester transfer protein (CETP), lecithin, or cholesterol acyltransferase (LCAT); capacity and/or rate for cholesterol binding; and capacity and/or rate for cholesterol transport.

The term “blockage due to lipid content” is measured as a percentage of a surface area of a cross-sectional slice of the artery and is used to refer to the extent that lateral flow through the artery is physically blocked.

Atheroma Diseases, Including Xanthomas

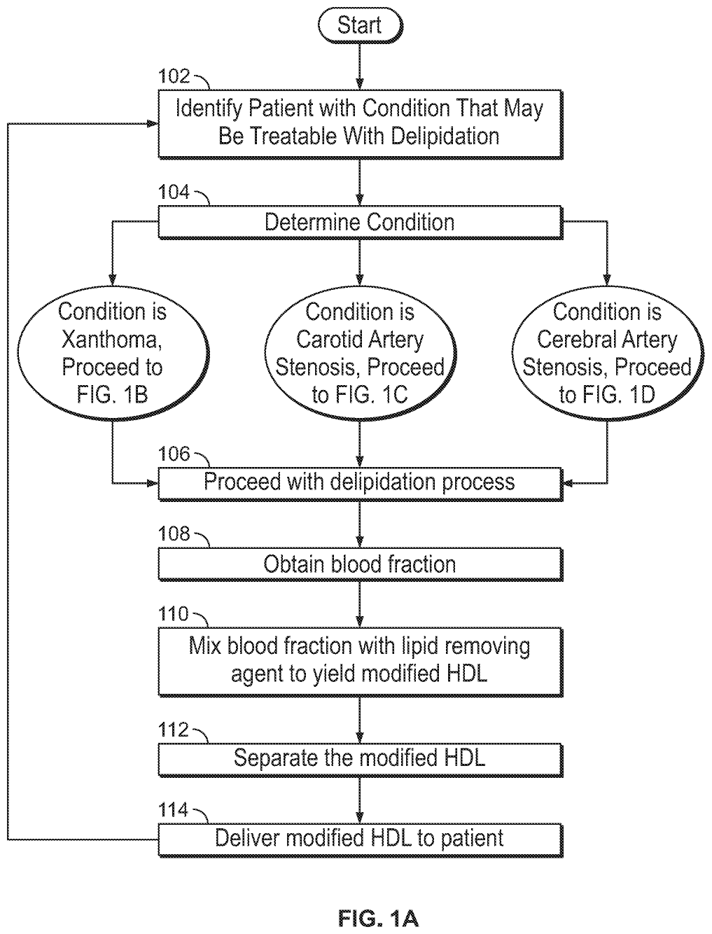

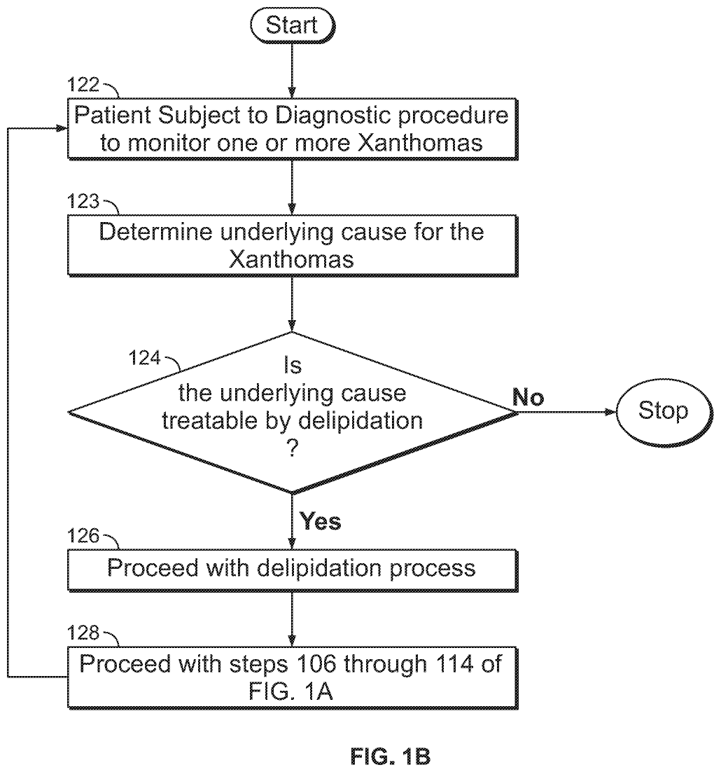

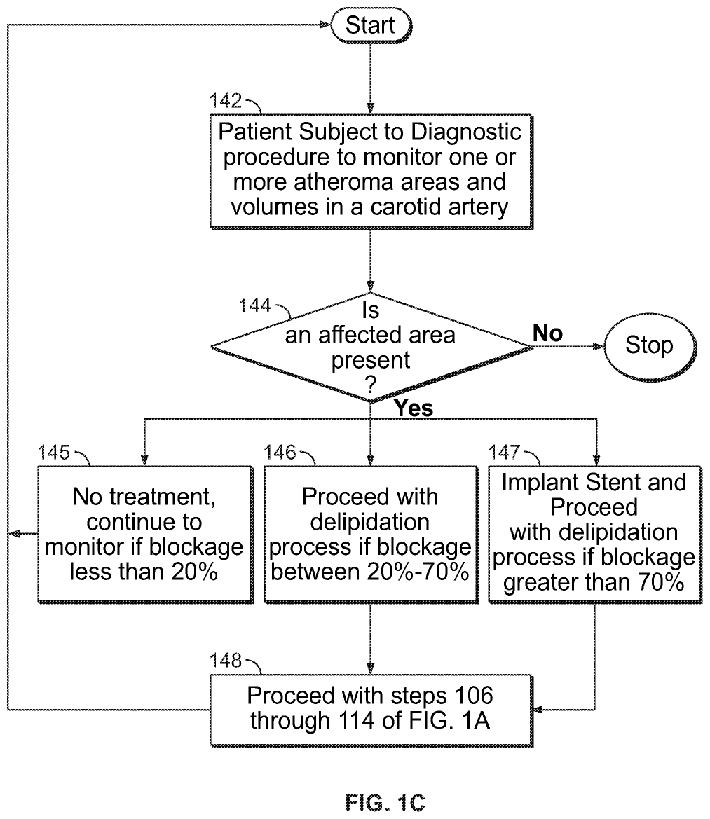

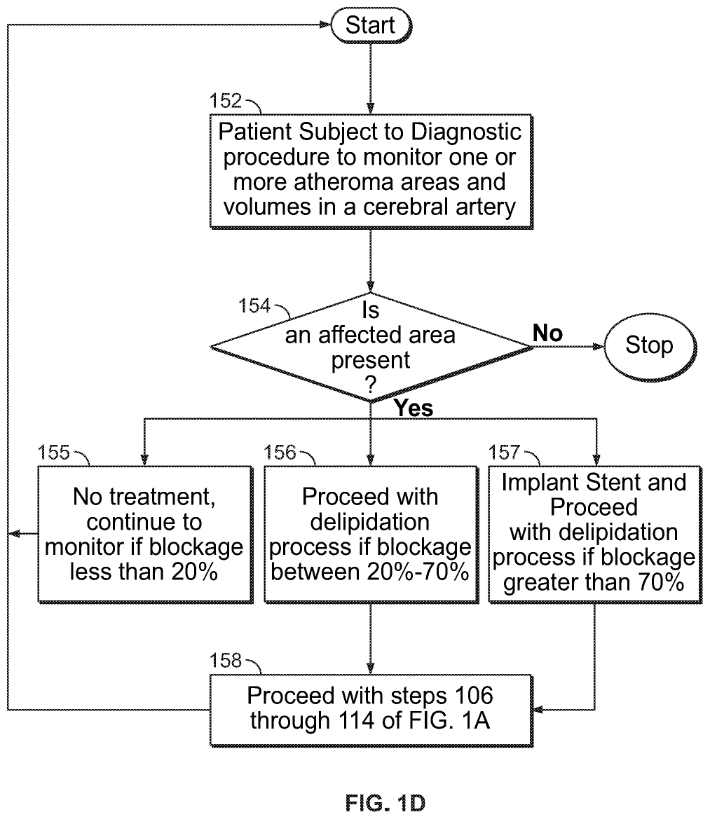

FIG. 1A is a flow chart illustrating an exemplary process of treating diseases, such as, but not limited to HoFH, HeFH, xanthomas, ischemic stroke, and CAS, among others, in accordance with some embodiments of the present specification. At step 102, a subject or a patient is identified with a condition that may be treatable using the delipidation systems and methods of the present specification. In some embodiments, the conditions include, but are not limited to, xanthomas, carotid artery stenosis, and cerebral artery stenosis. Xanthomas may be detected by a physical examination, such as by observing the skin of a patient. In case of CAS, in an embodiment, advanced medical imaging techniques, such as, but not limited to Computer Tomography (CT) angiogram and/or Intravascular Ultrasound (IVUS), may be used to detect areas within the inner layer of artery walls where lipid-containing degenerative material may have accumulated. Accumulated degenerative material may include fatty deposits which may include mostly macrophage cells, or debris, containing lipids, calcium and a variable amount of fibrous connective tissue. Analysis from the imaging techniques may also be used to identify and therefore monitor volumes of lipid-containing degenerative material accumulated within the inner layer of artery walls. Lipid-containing degenerative material and non-lipid-containing degenerative material may swell in the artery wall, thereby intruding into the channel of the artery and narrowing it, resulting in restriction of blood flow.

An step 104, the condition is confirmed to determine which diagnostic and/or therapeutic steps should be taken based on the condition. In various embodiments, the patient is identified as having at least one xanthoma or with an extent or percentage arterial blockage caused by degenerative material (lipid-containing or non-lipid-containing). If the condition is a xanthoma, the method of FIG. 1B is followed to determine if the patient is a candidate for therapy. If the condition is carotid artery stenosis, the method of FIG. 1C is followed to determine if the patient is a candidate for therapy. If the condition is cerebral artery stenosis, the method of FIG. 1D is followed to determine if the candidate for therapy. In an embodiment, the physician identifies one or more arteries with stenosis that have a blockage of 20%-70% due to accumulated lipids, in order to implement treatment methods in accordance with the present specification. In an embodiment, the physician identifies a sizeable area of one or more xanthomas, in order to implement treatment methods in accordance with the present specification. In some embodiments, xanthomas of any size are considered for treatment methods in accordance with the present specification. At step 106, if a xanthoma of any size is detected, or if at least 20% blockage of a carotid or cerebral artery is detected (according to the flow charts of FIGS. 1B, 1C, and 1D), the patient is subjected to the delipidation process. In some embodiments, if the patient has additional risk factors, such as diabetes and high blood pressure, the patient is considered at greater risk of future events (stroke) and is treated more aggressively and initiated on the delipidation process more quickly than if the additional risk factors were not present.

At step 108, a blood fraction of the patient is obtained. The process of blood fractionation is typically done by filtration, centrifuging the blood, aspiration, or any other method known to persons skilled in the art. Blood fractionation separates the plasma from the blood. In one embodiment, blood is withdrawn from a patient in a volume sufficient to produce approximately 12 ml/kg of plasma based on body weight. The blood is separated into plasma and red blood cells using methods commonly known to one of skill in the art, such as plasmapheresis. Then the red blood cells are stored in an appropriate storage solution or returned to the patient during plasmapheresis. The red blood cells are preferably returned to the patient during plasmapheresis. Physiological saline is also optionally administered to the patient to replenish volume.

Blood fractionation is known to persons of ordinary skill in the art, and is performed remotely from the method described in context of FIG. 1A. During the fractionation, the blood can optionally be combined with an anticoagulant, such as sodium citrate, and centrifuged at forces approximately equal to 2,000 times gravity. The red blood cells are then aspirated from the plasma. Subsequent to fractionation, the cells are returned to the patient. In some alternate embodiments, low-density lipoprotein (LDL) is also separated from the plasma. Separated LDL is usually discarded. In alternative embodiments, LDL is retained in the plasma. In accordance with embodiments of the present specification, blood fraction obtained at step 108 includes plasma with high-density lipoprotein (HDL), and may or may not include other protein particles. In embodiments, autologous plasma collected from the patient is subsequently treated via an approved plasmapheresis device. The plasma may be transported using a continuous or batch process.

At step 110, the blood fraction obtained at 108 is mixed with one or more solvents, such as lipid removing agents. In an embodiment, the solvents used include either or both of organic solvents sevoflurane and n-butanol. In embodiments, the plasma and solvent are introduced into at least one apparatus for mixing, agitating, or otherwise contacting the plasma with the solvent. In embodiments, the solvent system is optimally designed such that only the HDL particles are treated to reduce their lipid levels and LDL levels are not affected. The solvent system includes factoring in variables such as solvent employed, mixing method, time, and temperature. Solvent type, ratios and concentrations may vary in this step. Acceptable ratios of solvent to plasma include any combination of solvent and plasma. In some embodiments, ratios used are 2 parts plasma to 1 part solvent, 1 part plasma to 1 part solvent, or 1 part plasma to 2 parts solvent. In an embodiment, when using a solvent comprising 95 parts sevoflurane to 5 parts n-butanol, a ratio of two parts solvent per one part plasma is used. Additionally, in an embodiment employing a solvent containing n-butanol, the present specification uses a ratio of solvent to plasma that yields at least 3% n-butanol in the final solvent/plasma mixture. In an embodiment, a final concentration of n-butanol in the final solvent/plasma mixture is 3.33%. The plasma and solvent are introduced into at least one apparatus for mixing, agitating, or otherwise contacting the plasma with the solvent. The plasma may be transported using a continuous or batch process. Further, various sensing means may be included to monitor pressures, temperatures, flow rates, solvent levels, and the like. The solvents dissolve lipids from the plasma. In embodiments of the present specification, the solvents dissolve lipids to yield treated plasma that contains modified HDL particles with reduced lipid content. The process is designed such that HDL particles are treated to reduce their lipid levels and yield modified HDL particles without destruction of plasma proteins or substantially affecting LDL particles.

Energy is introduced into the system in the form of varied mixing methods, time, and speed. At 112, bulk solvents are removed from the modified HDL particles via centrifugation. In embodiments, any remaining soluble solvent is removed via charcoal adsorption, evaporation, or hollow fiber contractors (HFC) pervaporation. The mixture is optionally tested for residual solvent via use of gas chromatography (GC) or similar means. The test for residual solvent may optionally be eliminated based on statistical validation.

At 114, the treated plasma containing modified HDL particles with reduced lipid content, which was separated from the solvents at 112, is treated appropriately and subsequently returned to the patient. The modified HDL particles are HDL particles with an increased concentration of pre-beta HDL. Concentration of pre-beta HDL is greater in the modified HDL, relative to the original HDL that was present in the plasma before treating it with the solvent. The resulting treated plasma containing the HDL particles with reduced lipid and increased pre-beta concentration is optionally combined with the patient's red blood cells, if the red cells were not already returned during plasmapheresis, and administered to the patient. One route of administration is through the vascular system, preferably intravenously.

In embodiments, the patient is monitored again for changes in the previously monitored atheroma areas and volumes, specifically for lipid-containing degenerative material. Therefore the process is repeated from step 104, as described above. In embodiments, the patient is monitored repeatedly within a period of three to six months. The treatment cycle is also repeated at this frequency until the monitoring suggests substantially or completely enhanced cholesterol efflux. In an embodiment, when the atheroma area and volume are monitored to be below a threshold, the patient may be considered to have been treated and may not require further repetition of the treatment cycle. In embodiments, the treatment is considered effective when a size reduction of 10-100% is observed in the plaque causing the xanthomas or when artery stenosis is reduced by a range of 10-100%. In some embodiments, frequency of treatment may vary depending on the volume to be treated and the severity of the condition of the patient.

Xanthomas

FIG. 1B is a flow chart illustrating an exemplary process of treating just xanthomas, in accordance with some embodiments of the present specification. At step 122, a subject of a patient is diagnosed with one or more xanthomas. In some embodiments, a patient must be diagnosed with at least one xanthoma to be a candidate for treatment with the systems and methods of the present specification. In other embodiments, a patient must be diagnosed with more than one xanthoma to be a candidate for treatment with the systems and methods of the present specification. In still other embodiments, a patient must be diagnosed with more than one, more than two, more than three, or more than ten xanthomas, or any increment therein, to be a candidate for treatment with the systems and methods of the present specification. Xanthomas may be detected by a physical examination, such as by observing the skin of the patient.

For purposes of the present specification, a patient is screened for at least one xanthoma located on the patient's body. At step 123, an underlying cause for the xanthomas may be determined. While it is known that xanthomas are caused by high levels of fats, one or more advanced imaging techniques may be used to determine an underlying cause. In some embodiments, a xanthoma is biopsied to confirm the xanthoma is lipid filled and therefore, a result of a disease exhibiting dyslipidemia and therefore treatable by the systems and methods of the present specification. Some of the underlying causes includes familial hypercholesterolemia (FH), hyperlipoproteinemia, hyperlipoproteinemia type IV, familial combined hyperlipidemia, diabetes, hypothyroidism, and pancreatitis, among others. Of these, familial hypercholesterolemia (FH), hyperlipoproteinemia, hyperlipoproteinemia type IV, and familial combined hyperlipidemia may be treated with delipidation. Advanced medical imaging techniques, such as, but not limited to computer tomography (CT) angiogram and/or intravascular ultrasound (IVUS), may be used to detect areas within the inner layer of artery walls where lipid-containing degenerative material may have accumulated. Accumulated degenerative material may include fatty deposits which may include mostly macrophage cells, or debris, containing lipids, calcium and a variable amount of fibrous connective tissue. In some embodiments, one or more detected xanthomas are further characterized based on their presentation.

For example, in some embodiments, a xanthoma is characterized based on a threshold level of inflammation or based on a threshold level of scarring. In some embodiments, the level of inflammation is evaluated using a biopsy or observing a vessel wall by imaging to determine inflammation in the xanthoma. In some embodiments, any inflammation observed under direct visualization after biopsy or vessel wall imaging indicates the patient is a candidate for delipidation. In some embodiments, changes in plasma biomarkers for inflammation, including high sensitivity C reactive protein, interleukin 18 (IL 18), and tumor necrosis factor alpha, are used to monitor inflammation. In some embodiments, a patient having a xanthoma and presenting with a high sensitivity C reactive protein value greater than 1 mg/dl, an IL 18 level greater than 32 pg/ml, or a tumor necrosis factor alpha level greater than 11 is considered a candidate for delipidation. In some embodiments, a threshold level of scarring is patient driven and based on patient complaints, in conjunction with imaging and physical examination. In some embodiments, a patient complaint of an unsightly xanthoma along with confirmation on physical exam indicates the patient is a candidate for delipidation. Analysis from the imaging techniques may also be used to identify and therefore monitor volumes of lipid-containing degenerative material accumulated within the inner layer of artery walls. Lipid-containing degenerative material and non-lipid-containing degenerative material may swell in the artery wall, thereby intruding into the channel of the artery and narrowing it, resulting in restriction of blood flow. Therefore, in some embodiments, patients presenting with cutaneous xanthomas are also evaluated for unrecognized significant cardiovascular disease which is frequently associated with xanthomas.

Based on analysis from the diagnostic technique, in step 124, the presence and type of underlying condition that caused the at least one xanthoma is confirmed. It may be determined, from experience and knowledge, whether the underlying condition may be treated by a delipidation process for removing lipids from the blood of the patient. Xanthomas that are caused due to dyslipidemia are treatable by delipidation methods, such as those discussed in embodiments of the present specification. Diagnostic methods may be used for determining the type of dyslipidemia, such as familial or hyperlipidemia, to pinpoint as an underlying cause of the xanthomas, so that the xanthoma may be treated by the delipidation process of the present specification. The patient is identified with at least one xanthoma caused by the presence of degenerative material (lipid-containing or non-lipid-containing). If the condition may not be treated by the delipidation process, at step 124, the process is stopped. Thus, in an embodiment, the physician identifies presence of one or more xanthomas, in order to implement treatment methods in accordance with the present specification. At step 126, if it is determined at step 124 that the xanthoma is caused by a condition that may be treated by the delipidation processes of the present specification, the patient is subjected to the delipidation process. A delipidation process, from step 108 to step 114 as explained in context of FIG. 1A, is then performed at step 128.

In embodiments, the patient is monitored again for changes in the previously diagnosed xanthoma(s), for example, changes in size. Therefore the process is repeated from step 122, as described above. In some embodiments, frequency of treatment may vary depending on the volume to be treated and the severity of the condition of the patient.

In embodiments, the process described in the context of FIG. 1B is repeatedly performed for the xanthoma patient. In some embodiments, the process is repeated at least twice, based on a physical examination of the patient in order to observe the change in size of the one or more xanthomas. In some embodiments, the process is repeated once a week for at least two consecutive weeks. In still other embodiments, the process is repeated once a week for at least three, four, five, six, or seven weeks. The patient is monitored again for changes in the previously monitored xanthoma, and, in some embodiments, specifically for lipid-containing degenerative material. In some embodiments, a repeated diagnosis for xanthomas may be made by physical examination only. Therefore the process is repeated from step 122, as described above. In some embodiments, the treatment is repeated once a week for several weeks, such as for example for seven weeks. In embodiments, the patient is monitored repeatedly within a period of three to six months. The treatment cycle is also repeated at this frequency until the monitoring suggests substantially or completely enhanced cholesterol efflux and visible reduction or removal of xanthomas.

In an embodiment, when xanthomas are decreased by at least 5% in volume over the entire course of the treatment, the patient may be considered to have been treated and may not require further repetition of the treatment cycle. In various embodiments, the one or more xanthomas decrease in volume by a range of 5% to 100%, and every increment within, as a result of treatment over the entire treatment cycle. The size may be characterized by a surface area, a volume, and/or a periphery length of the xanthoma. In some embodiments, frequency of treatment may vary depending on the volume to be treated and the severity of the condition of the patient. In most cases, a reduction in the volume of the xanthomas is marked by a visible reduction in the size of the xanthomas. The location, composition and the color of xanthoma(s) may also change as a result of the decrease in the xanthoma volume. In patients with a lighter skin tone, the skin on or near the xanthoma area tends to have an orange and/or yellow tone. In some embodiments, especially for patients with a lighter skin tone, a positive change may be indicated where a color of the xanthoma is closer to the natural skin tone of the patient. Therefore, as there is improvement, the orange or yellow tone fades to a lighter shade and is closer to the natural skin tone. In some embodiments, physical examination in combination with biopsy is used to determine the change in xanthomas. In patients with a darker skin tone, it may be sufficient to diagnose an improvement based on the size and feel of the xanthoma. In embodiments, visible decrease in volume of xanthomas is observed with a decrease in HDL. In one example, a decrease in volume of 20-25% of xanthoma on the hand of a patient may indicate a successful treatment. In another example, a decrease in volume of about 10% of xanthoma on the Achilles tendon of a patient may indicate a successful treatment. In children, any decrease in size and/or volume of the xanthoma may indicate a successful treatment.

In embodiments, size of the xanthoma may be monitored with the help of one or more of available methods, such as but not limited to, laser lines guides, 3D scanners, or any other type of wound measurement/monitoring devices. In some embodiments, a durometer to measure hardness of human tissue may be used to assess change in the composition and therefore texture and hardness of the xanthoma. In some embodiments, a change in durometer value of at least 10% indicates improvement of the xanthoma and a positive response to treatment. In some embodiments, a reduction of at least 10% indicates improvement of the xanthoma and a positive response to treatment.

Carotid Artery Stenosis (CAS)

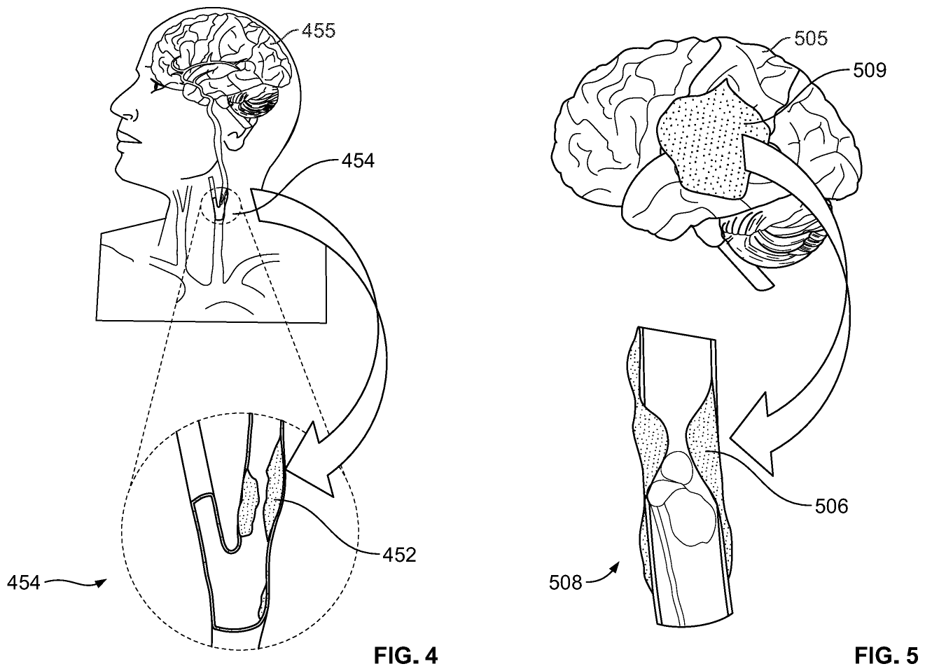

Referring to FIG. 1C, at step 142, a subject or a patient is subjected to a diagnostic procedure to monitor one or more atheroma areas and volumes in a carotid artery. CAS, also known as carotid atherosclerosis (CA), is said to occur when the blockage is in one of the brain's larger blood-supplying arteries such as a carotid artery. During a medical examination, a physician may be able to diagnose a possibility of carotid artery disease based on abnormal sounds, called bruits, with the help of a stethoscope. Physicians may also use tests to diagnose carotid stenosis, such as carotid ultrasound, magnetic resonance angiography (MRA), computerized tomography angiography (CTA), and cerebral angiography (carotid angiogram). Based on one or more tests, CAS patients are identified as having plaque in the carotid artery. Sometimes the CAS patients are asymptomatic. However, the affected artery may rupture in a manner similar to how plaque can rupture in a coronary artery. FIG. 4 illustrates plaque 452 in a carotid artery 454 of a patient 455. The illustrated blockage can potentially result in stroke, and the patient 455 is a candidate for therapy in accordance with the embodiments of the present specification. In some embodiments, the patient 455 is a candidate for therapy in accordance with the systems and methods of the present specification if the plaque 452 creates at least 20% stenosis of the carotid artery 454. The atherosclerosis within the artery wall results in reduced blood flow to the brain, often resulting in stroke. Acute strokes, also common in the elderly, may also be caused by an increase in cholesterol levels and metabolic disorders such as familial hypercholesterolemia. Ischemic stroke occurs when there is a blockage in an artery leading to the brain, and may be a secondary condition caused by a hemorrhagic stroke. Among the different types of ischemic stroke is a thrombotic stroke, which occurs when diseased or damaged cerebral arteries become blocked by the formation of a blood clot within the brain. Sometimes the blockage is in one of the brain's larger blood-supplying arteries such as the carotid or middle cerebral. Carotid stenosis is a vascular risk factor that may result in cerebral amyloid angiopathy (CAA), which is a condition caused by deposits of amyloid proteins in the wall or perivascular space of blood vessels in a brain. Carotid stenosis risk increases significantly with abnormal lipids or high cholesterol. Plaque usually builds up in the carotid arteries until the occurrence of a stroke.

Referring again to FIG. 1C, based on analysis from the diagnostic technique, in step 144, the presence and type of degenerative material is confirmed, and the extent or percentage of degenerative material (lipid-containing or non-lipid-containing) is determined. The process is stopped if no degenerative material is detected, or if the level of degenerative material is below a predetermined threshold or falls outside of a predetermined range of values. At step 145, in some embodiments, if less than 20% blockage is detected, the patient is not a candidate for delipidation treatment and the patient is continually monitored beginning at step 142. In an embodiment, the physician identifies one or more arteries with stenosis that have a blockage of 20%-70% due to accumulated lipids, in order to implement treatment methods in accordance with the present specification. At step 146, if at least 20% blockage of a carotid artery is detected and no more than 70% blockage is detected, the patient is subjected to the delipidation process. At step 147, if more than 70% blockage is detected, the patient receives a stent in the affected artery and the patient is subjected to the delipidation process. A delipidation process, from step 108 to step 114 as explained in context of FIG. 1A, is then performed at step 148.

Embodiments of the present specification, as described above in context of FIG. 1A and FIG. 1C, are performed to treat patients with CAS. In various embodiments, patients are treated one to two times per week for a duration of 1 to 14 weeks. A patient may be considered to be treated based on changes in the carotid arteries, such as reduced inflammation of the muscle or muscle wall of the carotid arteries based on carotid artery imaging, carotid artery biopsy, and plasma inflammatory biomarker levels. In some embodiments, a reduction of at least 10% in inflammation of the muscle or muscle wall of the carotid arteries is indicative of effective treatment. In some cases, diameter of the carotid artery is observed for changes to determine the effect of the treatment and observe the reduction in plaque size, in accordance with the embodiments of the present specification. In some cases, a change in the thickness of the carotid arterial wall of about 15%, preferably 25% or any increment therein, may indicate a successful treatment of the patient. In most cases, a 10-100% reduction in the plaque size within the carotid arteries, as measured subsequently during and after the treatment by methods such as CT Angiography, may be considered successful.

Different types of treatments may be provided depending on the diagnostic results and threshold values. In some cases, a stent may be used alternatively, or together in combination with the delipidation method of treatment of the present specification. At this stage, the physician may determine that either the treatment in accordance with embodiments of the present specification is not required as the disease has subsided, is not present, is not sufficient, or has been treated; or an alternative form of treatment is required.

In embodiments, the patient is monitored again for changes in the previously monitored atheroma areas and volumes in a carotid artery, specifically for lipid-containing degenerative material. Therefore the process is repeated from step 142, as described above. In embodiments, the patient is monitored repeatedly within a period of three to six months. The treatment cycle is also repeated at this frequency until the monitoring suggests substantially or completely enhanced cholesterol efflux. In some embodiments, frequency of treatment may vary depending on the volume to be treated and the severity of the condition of the patient.

Cerebral Atherosclerosis (CA)

Referring to FIG. 1D, at step 152, a subject or a patient is subject to a diagnostic procedure to monitor one or more atheroma areas and volumes in the cerebral arteries. CA is said to occur when the blockage is in one of the brain's larger blood-supplying arteries such as the middle cerebral. FIG. 5 illustrates plaque 506 in a middle cerebral artery 508 of a patient brain 505. The illustrated plaque 506 deposit can potentially result in a stroke, resulting, in some cases, in a loss of blood supply to a portion 509 of the patient's brain 505. As such, a patient with cerebral artery stenosis may be a candidate for therapy in accordance with the embodiments of the present specification. In some embodiments, the patient is a candidate for therapy in accordance with the systems and methods of the present specification if the plaque 506 creates at least 20% stenosis of the cerebral artery 508. Physicians may use tests to diagnose CA, such as doppler ultrasound, magnetic resonance angiography (MRA), computerized tomography angiography (CTA), and cerebral angiography. Based on one or more tests, CA patients are identified as having plaque in one or more cerebral arteries. Sometimes the CA patients are asymptomatic. However, the affected artery may rupture in a manner similar to how plaque can rupture in a coronary artery. The atherosclerosis within the artery wall results in reduced blood flow within the brain, often resulting in stroke.

Based on analysis from the diagnostic technique, in step 154, the presence and type of degenerative material is confirmed, and the extent or percentage of degenerative material (lipid-containing or non-lipid-containing) is determined. The process is stopped if no degenerative material is detected, or if the level of degenerative material is below a predetermined threshold or falls outside of a predetermined range of values. At step 155, in some embodiments, if less than 20% blockage is detected, the patient is not a candidate for delipidation treatment and the patient is continually monitored beginning at step 152. In an embodiment, the physician identifies one or more arteries with stenosis that have a blockage of 20%-70% due to accumulated lipids, in order to implement treatment methods in accordance with the present specification. At step 154, if at least 20% blockage of a cerebral artery is detected and no more than 70% blockage is detected, the patient is subjected to the delipidation process. At step 157, if more than 70% blockage is detected, the patient receives a stent in the affected artery and the patient is subjected to the delipidation process. A delipidation process, from step 108 to step 114 as explained in context of FIG. 1A, is then performed at step 158.

Embodiments of the present specification, as described above in context of FIG. 1A and FIG. 1D, are performed to treat patients with CA. In various embodiments, patients are treated one to two times per week for a duration of 1 to 14 weeks. A patient may be considered to be treated based on changes in the cerebral arteries, such as reduced inflammation of the muscle or muscle wall of the cerebral arteries. based on cerebral artery imaging and plasma inflammatory biomarker levels. In some embodiments, a reduction of at least 10% in inflammation of the muscle or muscle wall of the cerebral arteries is indicative of effective treatment. In some cases, diameter of the cerebral artery is observed for changes to determine the effect of the treatment and observe the reduction in plaque size, in accordance with the embodiments of the present specification. In some cases, a change in the thickness of the cerebral arterial wall of about 15%, or preferably 25% or any increment therein, may indicate a successful treatment of the patient. In most cases, a 10-100% reduction in the plaque within the cerebral arteries, as measured subsequently during and after the treatment by methods such as CT Angiography, may be considered successful.

Different types of treatments may be provided depending on the diagnostic results and threshold values. In some cases, a stent may be used alternatively, or together in combination with the delipidation method of treatment of the present specification. At this stage, the physician may determine that either the treatment in accordance with embodiments of the present specification is not required as the disease has subsided, is not present, is not sufficient, or has been treated, or an alternative form of treatment is required.

In embodiments, the patient is monitored again for changes in the previously monitored atheroma areas and volumes in a cerebral artery, specifically for lipid-containing degenerative material. Therefore the process is repeated from step 152, as described above. In embodiments, the patient is monitored repeatedly within a period of three to six months. The treatment cycle is also repeated at this frequency until the monitoring suggests substantially or completely enhanced cholesterol efflux. In some embodiments, frequency of treatment may vary depending on the volume to be treated and the severity of the condition of the patient.

Patients diagnosed with xanthomas, CAS, or CA, and treatable by delipidation, or identified with arteries with lipid-containing atheroma area/volume, are subjected to the delipidation process in accordance with embodiments of the present specification. In this case, a blood fraction of the patient is obtained. The process of blood fractionation is typically done by filtration, centrifuging the blood, aspiration, or any other method known to persons skilled in the art. Blood fractionation separates the plasma from the blood. In one embodiment, blood is withdrawn from a patient in a volume sufficient to produce approximately 12 ml/kg of plasma based on body weight. The blood is separated into plasma and red blood cells using methods commonly known to one of skill in the art, such as plasmapheresis. Then the red blood cells are stored in an appropriate storage solution or returned to the patient during plasmapheresis. The red blood cells are preferably returned to the patient during plasmapheresis. Physiological saline is also optionally administered to the patient to replenish volume.

Blood fractionation is known to persons of ordinary skill in the art, and is performed remotely from the method described in context of FIG. 1A.

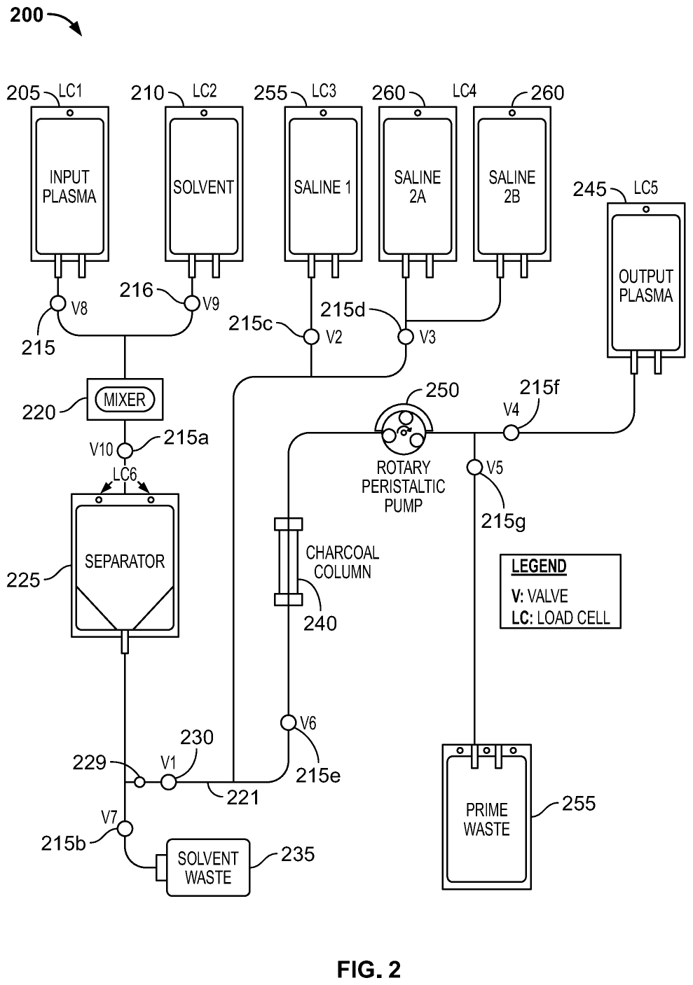

FIG. 2 illustrates an exemplary embodiment of a system and its components used to achieve the methods of the present specification. The figure depicts an exemplary basic component flow diagram defining elements of the HDL modification system 200. Embodiments of the components of system 200 are utilized after obtaining a blood fraction from a patient or another individual (donor). The plasma separated from the blood is brought in a sterile bag to system 200 for further processing. The plasma may be separated from blood using a known plasmapheresis device. The plasma may be collected from the patient into a sterile bag using standard apheresis techniques. The plasma is then brought in the form of a fluid input to system 200 for further processing. In embodiments, system 200 is not connected to the patient at any time and is a discrete, stand-alone system for delipidating plasma. The patient's plasma is processed by system 200 and brought back to the patient's location to be reinfused back into the patient. In alternate embodiments, the system may be a continuous flow system that is connected to the patient in which both plasmapheresis and delipidation are performed in an excorporeal, parallel system and the delipidated plasma product is returned to the patient.

A fluid input 205 (containing blood plasma) is provided and connected via tubing to a mixing device 220. A solvent input 210 is provided and also connected via tubing to mixing device 220. In embodiments, valves 215, 216 are used to control the flow of fluid from fluid input 205 and solvent from solvent input 210 respectively. It should be appreciated that the fluid input 205 contains any fluid that includes HDL particles, including plasma having LDL particles or devoid of LDL particles, as discussed above. It should further be appreciated that solvent input 210 can include a single solvent, a mixture of solvents, or a plurality of different solvents that are mixed at the point of solvent input 210. While depicted as a single solvent container, solvent input 210 can comprise a plurality of separate solvent containers. Embodiments of types of solvents that may be used are discussed above.

Mixer 220 mixes fluid from fluid input 205 and solvent from solvent input 210 to yield a fluid-solvent mixture. In embodiments, mixer 220 is capable of using a shaker bag mixing method with the input fluid and input solvent in a plurality of batches, such as 1, 2, 3 or more batches. An exemplary mixer is a Barnstead Labline orbital shaker table. In alternative embodiments, other known methods of mixing are utilized. Once formed, the fluid-solvent mixture is directed, through tubing and controlled by at least one valve 215a, to a separator 225. In an embodiment, separator 225 is capable of performing bulk solvent separation through gravity separation in a funnel-shaped bag.

In separator 225, the fluid-solvent mixture separates into a first layer and second layer. The first layer comprises a mixture of solvent and lipid that has been removed from the HDL particles. The first layer is transported through a valve 215b to a first waste container 235. The second layer comprises a mixture of residual solvent, modified HDL particles, and other elements of the input fluid. One of ordinary skill in the art would appreciate that the composition of the first layer and the second layer would differ based upon the nature of the input fluid. Once the first and second layers separate in separator 225, the second layer is transported through tubing to a solvent extraction device 240. In an embodiment, a pressure sensor 229 and valve 230 are positioned in the flow stream to control the flow of the second layer to solvent extraction device 240.

The opening and closing of valves 215, 216 to enable the flow of fluid from input containers 205, 210 may be timed using mass balance calculations derived from weight determinations of the fluid inputs 205, 210 and separator 225. For example, the valve 215b between separator 225 and first waste container 235 and valve 230 between separator 225 and solvent extraction device 240 open after the input masses (fluid and solvent) substantially balance with the mass in separator 225 and a sufficient period of time has elapsed to permit separation between the first and second layers. Depending on what solvent is used, and therefore which layer settles to the bottom of separator 225, either valve 215b between separator 225 and first waste container 235 is opened or valve 230 between separator 225 and solvent extraction device 240 is opened. One of ordinary skill in the art would appreciate that the timing of the opening is dependent upon how much fluid is in the first and second layers and would further appreciate that it is preferred to keep valve 215b between separator 225 and first waste container 235 open just long enough to remove all of the first layer and some of the second layer, thereby ensuring that as much solvent as possible has been removed from the fluid being sent to solvent extraction device 240.

In embodiments, an infusion grade fluid (“IGF”) may be employed via one or more inputs 260 which are in fluid communication with the fluid path 221 leading from separator 225 to solvent extraction device 240 for priming. In an embodiment, saline is employed as the infusion grade priming fluid in at least one of inputs 260. In an embodiment, 0.9% sodium chloride (saline) is employed. In other embodiments, glucose may be employed as the infusion grade priming fluid in any one of inputs 260.

A plurality of valves 215c and 215d are also incorporated in the flow stream from glucose input 255 and saline input 260 respectively, to the tubing providing the flow path 221 from separator 225 to solvent extraction device 240. IGF such as saline and/or glucose are incorporated into embodiments of the present specification in order to prime solvent extraction device 240 prior to operation of the system. In embodiments, saline is used to prime most of the fluid communication lines and solvent extraction device 240. If priming is not required, the IGF inputs are not employed. Where such priming is not required, the glucose and saline inputs are not required. Also, one of ordinary skill in the art would appreciate that the glucose and saline inputs can be replaced with other primers if required by the solvent extraction device 240.

In some embodiments, solvent extraction device 240 is a charcoal column designed to remove the specific solvent used in solvent input 210. An exemplary solvent extraction device 240 is an Asahi Hemosorber charcoal column, or the Bazter/Gambro Adsorba 300C charcoal column or any other charcoal column that is employed in blood hemoglobin perfusion procedures. A pump 250 is used to move the second layer from separator 225, through solvent extraction device 240, and to an output container 245. In embodiments, pump 250 is a rotary peristaltic pump, such as a Masterflex Model 77201-62.

The first layer is directed to waste container 235 that is in fluid communication with separator 225 through tubing and at least one valve 215b. Additionally, other waste, if generated, can be directed from the fluid path connecting solvent extraction device 240 and output container 245 to a second waste container 255. Optionally, in an embodiment, a valve 215f is included in the path from the solvent extraction device 240 to the output container 245. Optionally, in an embodiment, a valve 215g is included in the path from the solvent extraction device 240 to the second waste container 255.

In an embodiment of the present specification, gravity is used, wherever practical, to move fluid through each of the plurality of components. For example, gravity is used to drain input plasma 205 and input solvent 210 into mixer 220. Where mixer 220 comprises a shaker bag and separator 225 comprises a funnel bag, fluid is moved from the shaker bag to the funnel bag and, subsequently, to first waste container 235, if appropriate, using gravity.

In an additional embodiment, not shown in FIG. 2, the output fluid in output container 245 is subjected to a solvent detection system, or lipid removing agent detection system, to determine if any solvent, or other undesirable component, is in the output fluid. In embodiments, a solvent sensor is only employed in a continuous flow system. In one embodiment, the output fluid is subjected to sensors that are capable of determining the concentrations of solvents introduced in the solvent input, such as n-butanol or di-isopropyl ether. The output fluid is returned to the bloodstream of the patient and the solvent concentrations must be below a predetermined level to carry out this operation safely. In embodiments, the sensors are capable of providing such concentration information on a real-time basis and without having to physically transport a sample of the output fluid, or air in the headspace, to a remote device. The resultant separated modified HDL particles are then introduced to the bloodstream of the patient.

In one embodiment, molecularly imprinted polymer technology is used to enable surface acoustic wave sensors. A surface acoustic wave sensor receives an input, through some interaction of its surface with the surrounding environment, and yields an electrical response, generated by the piezoelectric properties of the sensor substrate. To enable the interaction, molecularly imprinted polymer technology is used. Molecularly imprinted polymers are plastics programmed to recognize target molecules, like pharmaceuticals, toxins or environmental pollutants, in complex biological samples. The molecular imprinting technology is enabled by the polymerization of one or more functional monomers with an excess of a crosslinking monomer in presence of a target template molecule exhibiting a structure similar to the target molecule that is to be recognized, i.e. the target solvent.

The use of molecularly imprinted polymer technology to enable surface acoustic wave sensors can be made more specific to the concentrations of targeted solvents and are capable of differentiating such targeted solvents from other possible interferents. As a result, the presence of acceptable interferents that may have similar structures and/or properties to the targeted solvents would not prevent the sensor from accurately reporting existing respective solvent concentrations.

Alternatively, if the input solvent comprises certain solvents, such as n-butanol, electrochemical oxidation could be used to measure the solvent concentration. Electrochemical measurements have several advantages. They are simple, sensitive, fast, and have a wide dynamic range. The instrumentation is simple and not affected by humidity. In one embodiment, the target solvent, such as n-butanol, is oxidized on a platinum electrode using cyclic voltammetry. This technique is based on varying the applied potential at a working electrode in both the forward and reverse directions, at a predefined scan rate, while monitoring the current. One full cycle, a partial cycle, or a series of cycles can be performed. While platinum is the preferred electrode material, other electrodes, such as gold, silver, iridium, or graphite, could be used. Although, cyclic voltammetric techniques are used, other pulse techniques such as differential pulse voltammetry or square wave voltammetry may increase the speed and sensitivity of measurements.

Embodiments of the present specification expressly cover any and all forms of automatically sampling and measuring, detecting, and analyzing an output fluid, or the headspace above the output fluid. For example, such automated detection can be achieved by integrating a mini-gas chromatography (GC) measuring device that automatically samples air in the output container, transmits it to a GC device optimized for the specific solvents used in the delipidation process, and, using known GC techniques, analyzes the sample for the presence of the solvents.

Referring back to FIG. 2, suitable materials for use in any of the apparatus components as described herein include materials that are biocompatible, approved for medical applications that involve contact with internal body fluids, and in compliance with U.S. PVI or ISO 10993 standards. Further, the materials do not substantially degrade from, for instance, exposure to the solvents used in the present specification, during at least a single use. The materials are sterilizable either by radiation or ethylene oxide (EtO) sterilization. Such suitable materials are capable of being formed into objects using conventional processes, such as, but not limited to, extrusion, injection molding and others. Materials meeting these requirements include, but are not limited to, nylon, polypropylene, polycarbonate, acrylic, polysulfone, polyvinylidene fluoride (PVDF), fluoroelastomers such as VITON, available from DuPont Dow Elastomers LLC., thermoplastic elastomers such as SANTOPRENE, available from Monsanto, polyurethane, polyvinyl chloride (PVC), polytetrafluoroethylene (PTFE), polyphenylene ether (PFE), perfluoroalkoxy copolymer (PFA), which is available as TEFLON PFA from E.I. du Pont de Nemours and Company, and combinations thereof.

Valves 215, 215a, 215b, 215c, 215d, 215e, 215f, 215g, 216 and any other valve used in each embodiment may be composed of, but are not limited to, pinch, globe, ball, gate or other conventional valves. In some embodiments, the valves are occlusion valves such as Acro Associates' Model 955 valve. However, the present specification is not limited to a valve having a particular style. Further, the components of each system described in accordance with embodiments of the present specification may be physically coupled together or coupled together using conduits that may be composed of flexible or rigid pipe, tubing or other such devices known to those of ordinary skill in the art.

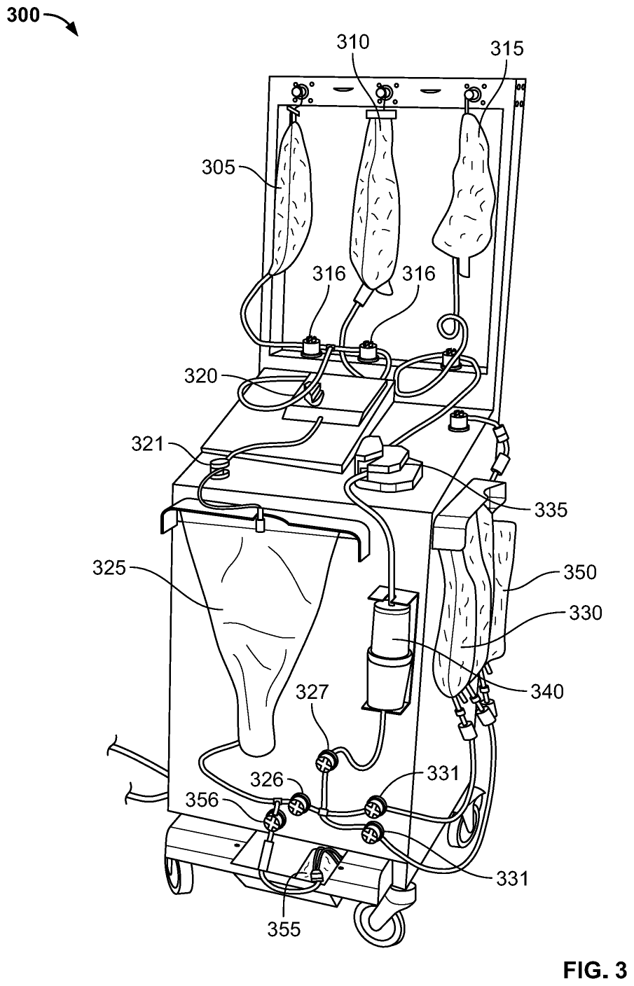

FIG. 3 illustrates an exemplary configuration of a system used in accordance with some embodiments of the present specification to achieve the processes disclosed herein. Referring to FIG. 3, a configuration of basic components of the HDL modification system 300 is shown. A fluid input 305 is provided and connected via tubing to a mixing device 320. A solvent input 310 is provided and also connected via tubing to a mixing device 320. Preferably valves 316 are used to control the flow of fluid from fluid input 305 and solvent from solvent input 310. It should be appreciated that the fluid input 305 preferably contains any fluid that includes HDL particles, including plasma having LDL particles or devoid of LDL particles, as discussed above. It should further be appreciated that solvent input 310 can include a single solvent, a mixture of solvents, or a plurality of different solvents that are mixed at the point of solvent input 310. While depicted as a single solvent container, solvent input 310 can comprise a plurality of separate solvent containers. The types of solvents that are used and preferred are discussed above.

The mixer 320 mixes fluid from fluid input 305 and solvent from solvent input 310 to yield a fluid-solvent mixture. Preferably, mixer 320 is capable of using a shaker bag mixing method with the input fluid and input solvent in a plurality of batches, such as 1, 2, 3 or more batches. Once formed, the fluid-solvent mixture is directed, through tubing and controlled by at least one valve 321, to a separator 325. In a preferred embodiment, separator 325 is capable of performing bulk solvent separation through gravity separation in a funnel-shaped bag.

In the separator 325, the fluid-solvent mixture separates into a first layer and second layer. The first layer comprises a mixture of solvent and lipid that has been removed from the HDL particles. The second layer comprises a mixture of residual solvent, modified HDL particles, and other elements of the input fluid. One of ordinary skill in the art would appreciate that the composition of the first layer and the second layer would differ based upon the nature of the input fluid. Once the first and second layers separate in separator 325, the second layer is transported through tubing to a solvent extraction device 340. Preferably, a pressure sensor 326 and valve 327 is positioned in the flow stream to control the flow of the second layer to the solvent extraction device 340.

Preferably, a glucose input 330 and saline input 350 is in fluid communication with the fluid path leading from the separator 325 to the solvent extraction device 340. A plurality of valves 331 is also preferably incorporated in the flow stream from the glucose input 330 and saline input 350 to the tubing providing the flow path from the separator 325 to the solvent extraction device 340. Glucose and saline are incorporated into the present specification in order to prime the solvent extraction device 340 prior to operation of the system. Where such priming is not required, the glucose and saline inputs are not required. Also, one of ordinary skill in the art would appreciate that the glucose and saline inputs can be replaced with other primers if the solvent extraction device 340 requires it.

The solvent extraction device 340 is preferably a charcoal column designed to remove the specific solvent used in the solvent input 310. An exemplary solvent extraction device 340 is an Asahi Hemosorber charcoal column. A pump 335 is used to move the second layer from the separator 325, through the solvent extraction device 340, and to an output container 315. The pump is preferably a peristaltic pump, such as a Masterflex Model 77201-62.

The first layer is directed to a waste container 355 that is in fluid communication with separator 325 through tubing and at least one valve 356. Additionally, other waste, if generated, can be directed from the fluid path connecting solvent extraction device 340 and output container 315 to waste container 355.

Preferably, an embodiment of the present specification uses gravity, wherever practical, to move fluid through each of the plurality of components. For example, preferably gravity is used to drain the input plasma 305 and input solvent 310 into the mixer 320. Where the mixer 320 comprises a shaker bag and separator 325 comprises a funnel bag, fluid is moved from the shaker bag to the funnel bag and, subsequently, to the waste container 355, if appropriate, using gravity.

In general, the present specification preferably comprises configurations wherein all inputs, such as input plasma and input solvents, disposable elements, such as mixing bags, separator bags, waste bags, solvent extraction devices, and solvent detection devices, and output containers are in easily accessible positions and can be readily removed and replaced by a technician.

To enable the operation of the above described embodiments of the present specification, it is preferable to supply a user of such embodiments with a packaged set of components, in kit form, comprising each component required to practice embodiments of the present specification. The kit may include an input fluid container (i.e. a high density lipoprotein source container), a lipid removing agent source container (i.e. a solvent container), disposable components of a mixer, such as a bag or other container, disposable components of a separator, such as a bag or other container, disposable components of a solvent extraction device (i.e. a charcoal column), an output container, disposable components of a waste container, such as a bag or other container, solvent detection devices, and, a plurality of tubing and a plurality of valves for controlling the flow of input fluid (high density lipoprotein) from the input container and lipid removing agent (solvent) from the solvent container to the mixer, for controlling the flow of the mixture of lipid removing agent, lipid, and particle derivative to the separator, for controlling the flow of lipid and lipid removing agent to a waste container, for controlling the flow of residual lipid removing agent, residual lipid, and particle derivative to the extraction device, and for controlling the flow of particle derivative to the output container.

In one embodiment, a kit comprises a plastic container having disposable components of a mixer, such as a bag or other container, disposable components of a separator, such as a bag or other container, disposable components of a waste container, such as a bag or other container, and, a plurality of tubing and a plurality of valves for controlling the flow of input fluid (high density lipoprotein) from the input container and lipid removing agent (solvent) from the solvent container to the mixer, for controlling the flow of the mixture of lipid removing agent, lipid, and particle derivative to the separator, for controlling the flow of lipid and lipid removing agent to a waste container, for controlling the flow of residual lipid removing agent, residual lipid, and particle derivative to the extraction device, and for controlling the flow of particle derivative to the output container. Disposable components of a solvent extraction device (i.e. a charcoal column), the input fluid, the input solvent, and solvent extraction devices may be provided separately.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method for treatment of at least one xanthoma in a patient wherein dyslipidemia is diagnosed as an underlying cause for the at least one xanthoma, comprising:
monitoring changes in at least one of size, volume, location, or composition of the at least one xanthoma;
determining if lipid-containing degenerative material is present in one or more blood vessels;
based on the determination of lipid-containing degenerative material and the monitoring of changes in at least one of size, volume, location, or composition of the at least one xanthoma, determining a treatment protocol for said at least one xanthoma, wherein the treatment protocol comprises administering to the patient a high density lipoprotein composition derived from mixing a blood fraction with a lipid removing agent; and
administering to the high density lipoprotein composition in accordance with the treatment protocol.

2. The method of claim 1, further comprising monitoring changes in the one or more blood vessels in the patient in order to determine if lipid-containing degenerative material is present in the one or more blood vessels.

3. The method of claim 1, wherein the high density lipoprotein composition is derived by:
obtaining the blood fraction from the patient;
mixing said blood fraction with the lipid removing agent to yield modified high-density lipoproteins;
separating said modified high-density lipoproteins; and
delivering said modified high-density lipoproteins to said patient.

4. The method of claim 3, wherein the modified high density lipoproteins have an increased concentration of pre-beta high density lipoprotein relative to high density lipoproteins from the blood fraction prior to mixing.

5. The method of claim 1, further comprising:
connecting the patient to a device for withdrawing blood;
withdrawing blood from the patient; and
separating blood cells from the blood to yield the blood fraction containing high density lipoproteins and low density lipoproteins.

6. The method of claim 1, further comprising repeating the administration to the patient of the high density lipoprotein composition.

7. The method of claim 1, further comprising, after said administration to the patient of the high density lipoprotein composition, monitoring changes in the at least one xanthoma by determining a change in a hardness of the at least one xanthoma.

8. The method of claim 7, wherein said change is determined by comparing a first hardness of the at least one xanthoma measured prior to said administration to a second hardness of the at least one xanthoma measured after said administration.

9. The method of claim 8, further comprising determining the first hardness and second hardness by applying a durometer to the at least one xanthoma.

10. The method of claim 8, further comprising repeating the administration to the patient of the high density lipoprotein composition until a durometer value of the second hardness is at least 10% less than a durometer value of the first hardness.

11. The method of claim 8, further comprising stopping the administration to the patient of the high density lipoprotein composition after a durometer value of the second hardness is at least 10% less than a durometer value of the first hardness.

12. The method of claim 1, further comprising determining the size of the at least one xanthoma using at least one of a laser lines guide, a 3D scanner, or a wound measurement device.

13. The method of claim 1, further comprising administering to the patient the high density lipoprotein composition only if a high sensitivity C reactive protein value of the patient exceeds a threshold value.

14. The method of claim 13, wherein the threshold value is greater than 1 mg/dl.

15. The method of claim 14, further comprising not administering to the patient the high density lipoprotein composition if the high sensitivity C reactive protein value of the patient does not exceed the threshold value.

16. The method of claim 1, further comprising administering to the patient a high density lipoprotein composition only if an interleukin 18 level of the patient exceeds a threshold value.

17. The method of claim 16, wherein the threshold value is greater than 32 pg/ml.

18. The method of claim 17, further comprising not administering to the patient the high density lipoprotein composition if the interleukin 18 level of the patient does not exceed the threshold value.

19. The method of claim 1, further comprising administering to the patient the high density lipoprotein composition only if a tumor necrosis factor alpha level of the patient exceeds a threshold value.

20. The method of claim 19, wherein the threshold value is greater than 11.

21. The method of claim 20, further comprising not administering to the patient the high density lipoprotein composition if the tumor necrosis factor alpha level of the patient does not exceed the threshold value.

22. A method for treatment of carotid artery stenosis in a patient, comprising:
monitoring changes in one or more blood vessels of the patient, wherein the one or more blood vessels comprise a carotid artery of the patient;
determining if lipid-containing degenerative material is present in said one or more blood vessels;
based on said monitoring and the determination of lipid-containing degenerative material, determining a treatment protocol for the carotid artery stenosis, wherein the treatment protocol comprises a placement of a stent in the patient and an administration to the patient of a composition derived from mixing a blood fraction of the patient with a lipid removing agent only if a blockage of the carotid artery of the patient exceeds 20% and does not exceed 70% of the carotid artery of the patient;
placing said stent; and
administering the high density lipoprotein composition to the patient in accordance with the treatment protocol.

23. The method of claim 22, wherein the composition is derived by:
obtaining the blood fraction from the patient;
mixing said blood fraction with the lipid removing agent to yield modified high-density lipoproteins;
separating said modified high-density lipoproteins; and
delivering said modified high-density lipoproteins to said patient.

24. The method of claim 23, wherein the modified high density lipoproteins have an increased concentration of pre-beta high density lipoprotein relative to high density lipoproteins from the blood fraction prior to mixing.

25. The method of claim 22, further comprising:
connecting the patient to a device for withdrawing blood;
withdrawing blood from the patient; and
separating blood cells from the blood to yield the blood fraction containing high density lipoproteins and low density lipoproteins.

26. The method of claim 22 further comprising repeating the method of treatment based on the monitoring changes in the one or more blood vessels of the patient.

27. The method of claim 22, further comprising repeating the administration to the patient of the high density lipoprotein composition.

28. The method of claim 22, further comprising, after said administration to the patient of the high density lipoprotein composition, monitoring changes in the carotid artery by determining a change in an inflammation level of a portion of the carotid artery.

29. The method of claim 28, wherein said change in the inflammation level is determined by using at least one of an imaging of the carotid artery, a biopsy of the carotid artery, or a measurement of a plasma biomarker level.

30. The method of claim 22, further comprising determining a first inflammation level measured prior to the administration to the patient of the high density lipoprotein composition and determining a second inflammation level measured after the administration to the patient of the high density lipoprotein composition.

31. The method of claim 30, further comprising repeating the administration to the patient of the high density lipoprotein composition until the measured second inflammation level exceeds a threshold reduction in value relative to the measured first inflammation level.

32. The method of claim 31, wherein the threshold reduction in value is 10%.

33. The method of claim 30, further comprising stopping the administration to the patient of the high density lipoprotein composition once the measured second inflammation level exceeds a threshold reduction in value relative to the measured first inflammation level.

34. The method of claim 33, wherein the threshold reduction in value is 10%.

35. The method of claim 22, further comprising administering to the patient of the high density lipoprotein composition at least one time per week for a duration of 1 to 14 weeks.

36. The method of claim 22, further comprising determining a first thickness in a wall of the carotid artery prior to the administration to the patient of the high density lipoprotein composition and determining a second thickness in the wall of the carotid artery measured after the administration to the patient of the high density lipoprotein composition.

37. The method of claim 36, further comprising repeating the administration to the patient of the high density lipoprotein composition until the determined second thickness exceeds a threshold reduction in value relative to the determined first thickness.

38. The method of claim 37, wherein the threshold reduction in value is 15%.

39. The method of claim 36, further comprising stopping the administration to the patient of the high density lipoprotein composition once the determined second thickness exceeds a threshold reduction in value relative to the determined first thickness.

40. The method of claim 39, wherein the threshold reduction in value is 15%.

41. A method for treatment of cerebral atherosclerosis in a patient, comprising:
monitoring changes in one or more blood vessels of the patient, wherein the one or more blood vessels comprise one or more cerebral arteries of the patient;
determining if lipid-containing degenerative material is present in said one or more blood vessels;
based on said monitoring and the determination of lipid-containing degenerative material, determining a treatment protocol for the cerebral atherosclerosis, wherein the treatment protocol comprises a placement of a stent in the patient and an administration to the patient of a composition derived from mixing a blood fraction of the patient with a lipid removing agent only if a blockage of the one or more cerebral arteries of the patient exceeds 20% and does not exceed 70% of the one or more cerebral arteries of the patient;
placing said stent; and
administering said composition only if a blockage of the one or more cerebral arteries of the patient exceeds 20% and does not exceed 70% of the one or more cerebral arteries of the patient.

42. The method of claim 41, wherein the composition is derived by obtaining the blood fraction from the patient;
mixing said blood fraction with the lipid removing agent to yield modified high-density lipoproteins;
separating said modified high-density lipoproteins; and
delivering said modified high-density lipoproteins to said patient.

43. The method of claim 42, wherein the modified high density lipoproteins have an increased concentration of pre-beta high density lipoprotein relative to high density lipoproteins from the blood fraction prior to mixing.

44. The method of claim 41, further comprising:
connecting the patient to a device for withdrawing blood;
withdrawing blood from the patient; and
separating blood cells from the blood to yield the blood fraction containing high density lipoproteins and low density lipoproteins.

45. The method of claim 41 further comprising repeating the method of treatment based on the monitoring changes in the one or more blood vessels of the patient.

46. The method of claim 41, further comprising repeating the administration to the patient of the high density lipoprotein composition.

47. The method of claim 41, further comprising, after said administration to the patient of the high density lipoprotein composition, monitoring changes in the one or more cerebral arteries by determining a change in an inflammation level of a portion of the one or more cerebral arteries.

48. The method of claim 47, wherein said change in the inflammation level is determined by using at least one of an imaging of the one or more cerebral arteries, a biopsy of the one or more cerebral arteries, or a measurement of a plasma biomarker level.

49. The method of claim 41, further comprising determining a first inflammation level measured prior to the administration to the patient of the high density lipoprotein composition and determining a second inflammation level measured after the administration to the patient of the high density lipoprotein composition.

50. The method of claim 49, further comprising repeating the administration to the patient of the high density lipoprotein composition until the measured second inflammation level exceeds a threshold reduction in value relative to the measured first inflammation level.

51. The method of claim 50, wherein the threshold reduction in value is 10%.

52. The method of claim 49, further comprising stopping the administration to the patient of the high density lipoprotein composition once the measured second inflammation level exceeds a threshold reduction in value relative to the measured first inflammation level.

53. The method of claim 52, wherein the threshold reduction in value is 10%.

54. The method of claim 41, further comprising administering to the patient of the high density lipoprotein composition at least one time per week for a duration of 1 to 14 weeks.

55. The method of claim 41, further comprising determining a first thickness in a wall of the one or more cerebral arteries prior to the administration to the patient of the high density lipoprotein composition and determining a second thickness in the wall of the one or more cerebral arteries measured after the administration to the patient of the high density lipoprotein composition.

56. The method of claim 55, further comprising repeating the administration to the patient of the high density lipoprotein composition until the determined second thickness exceeds a threshold reduction in value relative to the determined first thickness.

57. The method of claim 56, wherein the threshold reduction in value is 15%.

58. The method of claim 55, further comprising stopping the administration to the patient of the high density lipoprotein composition once the determined second thickness exceeds a threshold reduction in value relative to the determined first thickness.

59. The method of claim 58, wherein the threshold reduction in value is 15%.
